(19) United States
(12) Patent Application Publication
(10) Pub. No.: US 2012/0087859 A1
(43) Pub. Date: Apr. 12, 2012

(54) NANOCARRIER HAVING ENHANCED SKIN PERMEABILITY, CELLULAR UPTAKE AND TUMOUR DELIVERY PROPERTIES

(75) Inventors: Gi Yoong Tae, Gwangju (KR); Won II Choi, Gwangju (KR); Young Ha Kim, Gwangju (KR); Ja-Young Kim, Gwangju (KR); Jong Hyun Lee, Gwangju (KR)

(73) Assignee: GWANGJU INSTITUTE OF SCIENCE AND TECHNOLOGY, BUK-GU GEANGIU (KR)

(21) Appl. No.: 13/378,330

(22) PCT Filed: Jan. 21, 2011
(86) PCT No.: PCT/KR11/00449

§ 371 Date: Dec. 14, 2011

(30) Foreign Application Priority Data
Jan. 21, 2010 (KR) 10-2010-0005683
Jan. 19, 2011 (KR) 10-2011-0005553

(51) Int. Cl. A61K 47/36; A61K 38/43; A61K 49/00; A61K 51/06; A61K 49/12; A61K 38/02; A61P 35/00; A61K 31/70; A61K 31/7088; A61K 39/395; A61K 38/22; A61K 39/00; A61K 38/28; A61K 38/19; A61K 38/21; A61K 38/20; A61K 38/18; A61K 38/25; A61K 38/13; A61K 31/713; A61K 33/24; A61K 31/704; A61K 31/337; C08G 65/333; C08J 3/28; C08F 16/06; C08J 9/00; C08B 37/08; B82Y 99/00; B82B 3/00

(52) U.S. Cl. 424/1.11; 536/20; 424/94.1; 424/9.6; 424/9.3; 514/1.1; 514/19.3; 514/23; 514/44 R; 514/777; 514/772.2; 424/130.1; 424/135.1; 514/9.7; 424/184.1; 514/5.9; 424/85.1; 424/85.7; 424/85.6; 424/85.5; 424/85.2; 514/8.1; 514/8.2; 514/11.2; 514/20.5; 514/44 A; 424/649; 514/34; 514/274; 514/449; 525/54.2; 522/88; 521/50.5; 977/788; 977/896

(57) ABSTRACT

The present invention relates to a biopolymer-modified nanocarrier in which chitosan is bound to a water-soluble biocompatible polymer that has been crosslinked via a photo-crosslinkable functional group; wherein the chitosan-modified nanocarrier has a diameter which changes in accordance with changes in temperature, has enhanced skin permeability or cellular uptake and selective delivery to cancer tissue as compared with a bare nanocarrier to which chitosan has not been bound, and exhibits characteristics that are advantageous in photothermal therapy. The chitosan-modified nanocarrier of the present invention exhibits highly superior efficacy as a transdermal carrier, since the skin permeability is enhanced to a significant level as compared with a bare nanocarrier that has no chitosan. The chitosan-modified nanocarrier of the present invention can be advantageous in the imaging and photothermal therapy of tumour cells and cancer cells, since the cellular uptake by tumour cells and cancer cells is substantially improved.

### TECHNICAL FIELD

The present invention disclosed herein relates to a nanocarrier with enhanced skin permeability, cellular uptake and tumour delivery properties.

### BACKGROUND ART

Most of the nanoparticle systems used to deliver therapeutic proteins or drugs into the body are prepared by emulsion evaporation method using organic solvents.

However, these conventional methods need to include complicated steps, and also have problems associated with the use of organic solvents, such as cytotoxicity and an increasing cost for preparation (T. G. Park et al., Biomacromolecules 8 (2007) 650-656; T. G. Park et al., Biomacromolecules 7 (2006) 1864-1870; D. T. Birnbaum, et al., J. Control. Rel. 65 (2000) 375-387). Therefore, there have been extensive researchers focused on developing a novel method of preparing nanoparticles that can ensure the stability of drugs encapsulated inside nanoparticles.

In order to solve these problems, there have been attempts to use supercritical fluid, which are nontoxic solvents, for the preparation of nanoparticles. However, this process was not widely employed because most of the clinical polymers exhibit limited solubility in supercritical fluid (K. S. Soppimath et al., J. Control. Rel. 70 (2001) 1-20).

In addition, U.S. Pat. No. 5,019,400 discloses a process of preparing microspheres for protein drug delivery by spraying a biocompatible polymer, poly (D,L-lactic-co-glycolic acid) (referred to as “PLGA” hereinafter) into very cold temperature liquid to prepared micro particle for delivering protein drugs. However, there was a problem when using hydrophobic organic solvent for dissolving PLGA. Further, U.S. Pat. No. 6,586,011 discloses a process for preparing nanoparticle system for delivering protein by spraying into very cold temperature liquid. However, a cross-linking agent used for manufacturing nanoparticle seriously damages the stability of the protein drug.

Solvent evaporation method used for preparing nanoparticles also generates various problems associated with the use of organic solvent. Meanwhile, instead of using highly hydrophobic and toxic organic solvents, a salting-out method for preparing PLGA by using a water miscible organic solvent (e.g., action) has been reported. However, this method still has problems as lowered activity and stability of the protein drug (E. Allemann et al., Pharm. Res. 10 (1993) 1732-1737).

Furthermore, in regards to the modification or functionalization of one of the most popular natural polymer chitosan, Korea Patent No. 766820 discloses a method of improving the delivery of protein through mucosal barrier by functionalizing one type of polymer protein by chitosan. In addition, WO 2008/136773 discloses a functionalized nanoparticle, which surface is modified by chitosan that can be used for molecular imaging agent, biosensing agent and drug delivery system (DDS).

Topical and transdermal delivery of drug has many advantages including 1) continuous delivery of drug at constant rate, 2) reduce side effects, 3) improve treatment effect, 4) overcome the low bioavailability with oral administration, 5) reduce the dosing number and 6) easy to discontinue the drug administration when necessary.

However, the development of transdermal biomedical delivery reagent such as high molecular weight protein has not been successful.

Photothermal therapy (also called photothermal ablation), photothermal radiation or optical hyperthermia system are therapies gaining interest because of the low invasive treatment method for solid tumours (1-6). Generally, the technique that involves a step for converting the light absorbed by non-isotope mechanism into local heat has several advantages including 1) relatively simple use in cancer cell ablation, 2) fast recovery, 3) less side effect and 4) shorter hospitalization period (7). The use of near-infrared (NIR) spectrum has merits due to low absorption in normal tissue and has maximum penetration with high spatial accuracy without damaging the normal tissues (8-10).

Several nanostructures, including aggregated gold nanoparticles (11), gold nanosehells (12-14), gold nanocage (15), core-free AuAg dendrites (7), gold nanorod (GNR) (16-18) and carbon nanotube were NIR irradiated for photothermal cancer therapy. The plasma-resonance GNR has gained interest, since the light absorption range can be finely tuned by adjusting the aspect ratio. GNRs have other advantages including efficient large scale synthesis, easy functionalization, high photothermal inversion and colloidal stability (20-21). Despite the advantages, GNRs prepared by the seed-mediated synthesis have a bilayer capping of cetyltrimethylammonium bromide (CTAB) which shows cytotoxicity, thus limiting the clinical application (18). The surface modification of GNRs have been reported to reduce the cytotoxicity: e.g., phosphatidylcholine (PC)-modified GNRs, poly(sodium-4-styrenesulfonate)(PSS)-coated GNRs, GNR embedded complex nanoparticle and PEG treated GNRs, which showed lower cytotoxicity compared to CTAB-capped GNRs.

In photothermal cancer therapy, it is important to selectively deliver the GNRs to the target tumour. Aptamer-conjugated GNR and RGD-conjugated dendramer treated GNR have shown selective and effective target tumour therapy. The specific substrate conjugated GNR showed efficacy in in vivo photothermal cancer treatment. However this effect was limited in tumour-targeted photothermal therapy in animals (in vitro), which showed high localization of specific substrate conjugated GNR in liver tissue during blood circulation. High level of CTAB-stabilized GNR localized in the liver 0.5 hr after i.v. injection has been reported, probably due to the hard and rigid characteristics of the GNR (27). A technique of GRN PEGylation has been used (27) to overcome these limitations in tumour-targeted photothermal therapy in animals. However, the limited effect of photothermal cancer therapy may also be due to their fast excretion rate (half life of 1 hr). Therefore, a novel method for effectively delivering GNRs into the tumour site was highly anticipated.

Throughout this application, various publications and patents are referred and citations are provided in parentheses. The disclosures of these publications and patents in their entities are hereby incorporated by references into this application in order to fully describe this invention and the state of the art to which this invention pertains.

### DISCLOSURE

### Technical Problem

The present inventors carried out an extensive research to develop a temperature-sensitive nanocarrier with enhanced skin permeability, cellular uptake, selective delivery to cancer tissue and advantageous in photothermal therapy. As a result, production of a nanocarrier with the above improved characteristics was confirmed, when a nanocarrier is prepared from a water soluble biopolymer having a photo-crosslinkable functional group, and with chitosan, thereby completed the present invention.

Accordingly, it is an object of the present invention is to provide a nanocarrier with enhanced skin permeability, cellular uptake and tumour delivery properties and advantageous in photothermal therapy.

It is another object of the present invention to provide a composition for transdermal delivery.

It is still another object of the present invention to provide a composition for in vivo tumor or cancer imaging.

It is another object of the present invention to provide a composition for photothermal cancer therapy.

It is still another object of the present invention to provide a process for preparing a chitosan-modified nanocarrier with enhanced skin permeability, cellular uptake or tumour delivery properties.

Other objects and advantages of the present invention will become apparent from the detailed description to follow and together with the appended claims and drawings.

### Technical Solution

In one aspect of this invention, there is provided a biopolymer-modified nanocarrier in which chitosan is bound to a water-soluble biocompatible polymer that has been crosslinked via a photo-crosslinkable functional group at the end; wherein the chitosan-modified nanocarrier has a diameter which changes in accordance with changes in temperature, has enhanced skin permeability, cellular uptake, selective delivery to cancer tissue or increased photothermal effect as compared with a bare nanocarrier to which chitosan has not been bound.

The present inventors carried out extensive research to develop a thermo-sensitive nanocarrier with enhanced skin permeability, cellular uptake, selective delivery to cancer tissue and advantageous in photothermal therapy. As a result, production of a nanocarrier with the above improved characteristics was confirmed, when a nanocarrier is prepared from a water soluble biopolymer having a photo-crosslinkable functional group and with chitosan.

The term ‘a biocompatible polymer’ used herein refers to a polymer having the tissue compatibility and the blood compatibility so that it causes neither the tissue necrosis nor the blood coagulation upon contact with tissue or blood. The term ‘a water-soluble biocompatible polymer’ used herein refers to a biocompatible polymer soluble in water or water-miscible solvent (e.g., methanol, ethanol, acetone, acetonitrile, N,N-dimethylformamide and dimethylsulfoxide), preferably in water.

According to the preferred embodiment, examples of a water-soluble biocompatible polymer herein include poly(ethylene glycol), poly(ethylene oxide), poly(vinyl alcohol), poly(ethylene oxide)-poly(propylene oxide) block copolymer, alkylcellulose, hydroxyalkylcellulose, heparin, hyaluronic acid, chitosan, dextran or alginate. When a surfactant-like polymer comprising hydrophobic and hydrophilic parts is used among the water-soluble biocompatible polymer, it is preferred to additionally introduce hydrophobic parts to this polymer for achieving the aims of the present invention.

More preferably, a water-soluble biocompatible used herein is a poloxamer-based polymer.

More preferably, a water-soluble biocompatible polymer herein is a poloxamer-based polymer. Most preferably, a water-soluble biocompatible polymer herein is a polymer of Formula 1:

(PC1)-(PE)ₓ-(PPO)_{y}-(PE)_{z}-(PC2) Formula 1

wherein PE is ethylene oxide; PPO is propylene oxide; each of PC1 and PC2 is a photo-crosslinkable functional group; and each of x, y and z is independently an integer of 1-10,000.

A photo-crosslinkable functional group is preferred to exist at the both ends of a biocompatible polymer.

In a preferred embodiment, a photo-crosslinkable functional group comprises a C═C double bond.

Preferable examples of a photo-crosslinkable functional group include but are not limited to acrylate, diacrylate, oligoacrylate, methacrylate, dimethacrylate, oligomethacrylate, coumarin, thymine and cinnamate, more preferably acrylate, diacrylate, oligoacrylate, methacrylate, dimethacrylate and oligomethacrylate, most preferably acrylate.

A water-soluble biocompatible polymer crosslinked to a photo-crosslinkable functional group is modified by compatible chitosan.

Examples of compatible chitosan to modify the water-soluble biocompatible polymer may include chitosan known in the prior art, preferably any one or a combination of two or more selected from chitosan, heparin, alginate, hyaluronic acid, chondroitin sulfate, dermatan 5-sulfate, keratan sulfate, cellulose, hemi-cellulose, carboxymethly cellulose, dextran and dextran sulfate, poly(ethylene imine) and polylysine, most preferably, chitosan.

According to the preferred embodiment, the chitosan is bound to the water-soluble biocompatible polymer via a photo-crosslinkable functional group. A photo-crosslinkable functional group existing in chitosan is as described above.

The most preferably example of chitosan used to modify the biocompatible polymer is one of the most abundant organic polymers in nature next to cellulose. Chitosan is produced by deacetylation of chitin, found in crustacean species such as crab and shrimp, insect species such as grasshopper and dragonfly, mushroom species such as flammulina velutipes and lentinula edodes and cell walls of fungi. Chitosans are produced by deacetylation of the amine residue of chitin, which is formed by linear linkage of N-acetyl-D-glucosamine monomers (Errington N, et al., Hydrodynamic characterization of chitosan varying in molecular weight and degree of acetylation. Int J Biol Macromol. 15: 1123-7 (1993)). When compared with chitin, chitosan exists as polycation in acidic solution because of the deacetylation of amine residue. Thus, chitosan is molded into different forms such as powder, fiber, thin film, gel and bead since the water solubility increases in acid solution, and has good processability and high mechanical strength after drying (E. Guibal, et al., Ind. Eng. Chem. Res., 37: 1454-1463 (1998)). Chitosan can be categorized into oligomer composed of about 12 monomer units and high molecular weight, polymers. Chitosan polymer can be divided into low-molecular-weight-chitosan having molecular weight less than 15 Da, high-molecular-weight-chitosan with molecular weight of 70,000˜100,000 Da, and medium-molecular-weight-chitosan having the size range in the middle. Chitosan is widely applied in industries and clinical areas because of their stable, environmentally friendly, biodegradable and highly biocompatible characteristics. Also, chitosan is safe is known to not induce any immunogenic side effects. In the body, chitosan is degraded into N-acetylglucosamine by lysozyme, and then used for glycoprotein synthesis and excreted as carbon dioxide (Chandy T. Sharma C P. Chitosan as a biomaterial. Biomat Art Cells Art Org. 18:1-24 (1990)).

The present invention used highly biocompatible chitosan along with other biocompatible polymers as a carrier, and showed enhanced effect when the chitosan-modified nanocarrier was used as transdermal reagent or cancer targeting molecule.

The chitosan used in the present invention may be any conventional chitosan, preferably molecular weight of 500-20,000 Da. There is a problem of weak carrier function when the molecular weight is below 500 Da, and chitosan self-aggregates when the molecular weight is higher than 20,000 Da. Preferably, the chitosan is an oligomer.

In a preferred embodiment, the average diameter of chitosan-modified nanocarriers herein increases as temperature decreases, whereas the average diameter decreases as temperature increases. In a more preferred embodiment, the average diameter of chitosan-modified nanocarriers measured at 4° C. is 3-20 times, more preferably 4-15 times, still more preferably 5-12 times, most preferably 7-10 times, bigger than that measured at 40° C.

The modulation of average diameter of chitosan-modified nanocarriers herein is reversible in response to temperature change.

A pore size in chitosan-modified nanocarriers changes depending on the diameter of the chitosan-modified nanocarriers. After drugs to be delivered are encapsulated inside enlarged pores of chitosan-modified nanocarriers at a lower temperature, for example 4° C., the administration of the chitosan-modified nanocarriers into a human body decreases the pore size, thereby enabling the sustained release of the drugs.

In a preferred embodiment, chitosan-modified nanocarriers herein have a pore size of 3-20 nm, more preferably 3-15 nm, most preferably 5-10 nm when measured at 37° C.

In a preferred embodiment, the chitosan-modified nanocarrier is dispersed in an aqueous dispersion phase. In a preferred embodiment, the pore size of the chitosan-modified nanocarrier at 37° C. is 3 to 20 mm.

In a preferred embodiment, the chitosan-modified nanocarriers are not hydrogel but nanoparticulate. As ascertained in Examples herein, chitosan-modified nanocarriers herein are round-shaped nanoparticles. In a preferred embodiment, nanocarriers have an average diameter of 50-500 nm, more preferably 100-400 nm, most preferably 120-300 nm. In another preferred embodiment, nanoparticles herein are preferred to have an average diameter of 200 nm or less so that the sterilization of the final chitosan-modified nanocarriers may be conveniently conducted by using a sterile filter. Chitosan-modified nanocarriers herein are preferred to have a polydispersity of 0.1 or less because a polydispersity of 0.1 or less is considered as a stable monodispersity. More preferably, chitosan-modified nanocarriers herein have a polydispersity of 0.01-0.1.

Various therapeutically effective materials can be delivered by chitosan-modified nanocarriers of the present invention without limitation. In a preferred embodiment, examples of a material to be delivered in the present invention include a protein, a peptide, a nucleic acid, a saccharide, a lipid, a nanoparticle, a compound, an inorganic compound and a fluorescent material.

Examples of a protein or peptide that can be delivered by chitosan-modified nanocarriers of the present invention include but are not limited to a hormone, a hormone analog, an enzyme, an enzyme inhibitor, a signaling protein or segments thereof, an antibody or segments thereof, a single-chain antibody, a binding protein or a binding domain thereof, an antigen, an attachment protein, a structural protein, a regulatory protein, a toxoprotein, a cytokine, a transcription regulatory factor, a blood clotting factor and a vaccine. Specific examples of a protein or peptide that can be delivered by a drug delivery system herein include, without limitation, insulin, IGF-1 (insulin-like growth factor 1), a growth hormone, erythropoietin, G-CSFs (granulocyte-colony stimulating factors), GM-CSFs (granulocyte/macrophage-colony stimulating factors), interferon alpha, interferon beta, interferon gamma, interleukin-1 alpha and beta, interleukin-3, interleukin-4, interleukin-6, interleukin-2, EGFs (epidermal growth factors), calcitonin, VEGF (vascular endothelial cell growth factor), FGF (fibroblast growth factor), PDGF (platelet-derived growth factor), ACTH (adrenocorticotropic hormone), TGF-β (transforming growth factor beta), BMP (bone morphogenetic protein), TNF (tumour necrosis factor), atobisban, buserelin, cetrorelix, deslorelin, desmopressin, dynorphin A (1-13), elcatonin, eleidosin, eptifibatide, GHRH-II (growth hormone releasing hormone-II), gonadorelin, goserelin, histrelin, leuprorelin, lypressin, octreotide, oxytocin, pitressin, secretin, sincalide, terlipressin, thymopentin, thymosine α 1, triptorelin, bivalirudin, carbetocin, cyclosporine, exedine, lanreotide, LHRH (luteinizing hormone-releasing hormone), nafarelin, parathormone, pramlintide, T-20 (enfuvirtide), thymalfasin and ziconotide.

Examples of a nucleic acid that can be delivered by the chitosan-modified nanocarrier herein include, without limitation, a DNA, a DNA aptamer, a RNA aptamer, a ribozyme, a miRNA, an antisense oligonucleotide, siRNA, shRNA, a plasmid and a vector (e.g., adenovirus vector, retrovirus vector).

The material that can be delivered by the chitosan-modified nanocarrier is preferably a drug, for non-limited example, including anti-inflammatory agent, pain killers, anti-arthritis agent, cholinergic agonist, anti-spasmodic agent, anti-depressant, anti-phsychotic drug, ataractic agent, anti-anxiety drug, narcotic analgesic drug, anti-Parkinson's disease drug, anti-tumour agent, anti-angiogenesis agent, immune suppressor, antivirus, antibiotic, anorectic agent, analgesia, anti-cholinergic agent, anti-hemicranin agent, anti-histamine agent, hormone agent, coronary, cerebrovascular and peripheral vasodilators, contraceptives, anti-thrombosis agents, diuretics, anti-hypertensive drugs, cardiovascular disease drug and cosmetic components (e.g., anti-wrinkle agent, anti-aging agent and skin whitening agent).

Most preferably, the drug that can be delivered by chitosan-modified nanocarrier is anti-tumour agent. Specific examples of an anti-tumour agent that can be delivered include, without limitation, cisplatin, carboplatin, procarbazine, mechlorethamine, cyclophosphamide, ifosfamide, melphalan, chlorambucil, bisulfan, nitrosourea, dactinomycin, daunorubicin, doxorubicin, bleomycin, plicomycin, mitomycin, etoposide, tamoxifen, taxol, transplatinum, 5-fluorouracil, adriamycin, vincristine, vinblastine and methotrexate.

Examples of a nanoparticle that can be delivered by the chitosan-modified nanocarrier herein include, without limitation, gold nanoparticles, silver nanoparticles, iron nanoparticles, transition metal nanoparticles and metal oxide nanoparticles (e.g., ferrite nanoparticles). Ferrite nanoparticles delivered by the chitosan-modified nanocarrier herein can be used as an imaging agent for MR (magnetic resonance).

When delivering a fluorescent material using chitosan-modified nanocarrier herein, preferably the fluorescent material is bound to the surface of the chitosan-modified nanocarrier. For example, the fluorescence material may be bound to a protein or metal nanoparticles (e.g., magnetic nanoparticles). Examples of a fluorescent material herein include but are not limited to fluorescein and derivatives thereof, rhodamine and derivatives thereof, Lucifer Yellow, B-phycoerythrin, 9-acridine isothiocyanate, Lucifer Yellow Vs, 4-acetamido-4′-isothio-cyanatostilbene-2,2′-disulfonic acid, 7-diethylamino-3-(4′-isothiocyanatophenyl)-4-methylcoumarin, succinimidyl-pyrene butyrate, 4-acetamido-4′-isothiocyanatostilbene-2,2′-disulfonic acid derivative, LC™-Red 640, LC™-Red 705, Cy5, Cy5.5, lysamine, isothiocyanate, erythrosin isothiocyanate, diethylenetriamine pentacetate, 1-dimethylaminonaphthyl-5-sulfonate, 1-anilino-8-naphthalene sulfonate, 2-p-touidinyl-6-naphthalene sulfonate, 3-phenyl-7-isocyanatocoumarin, 9-isothiocyanatoacridine, acridine orange, N-(p-(2-benzoxazolyl)phenyl)maleimide, benzoxadiazole, stilbene and pyrene.

In an preferred embodiment of the present invention, a protein, a peptide, a nucleic acid, a saccharide, a lipid, a compound, an inorganic compound or a fluorescent material to be delivered by the nanocarrier has high molecular weights.

In an embodiment of the present invention, one of the features is that a material to be delivered can be spontaneously encapsulated inside chitosan-modified nanocarriers simply by mixing the nanocarriers and the material to be delivered. That is, a material to be delivered can be spontaneously loaded on chitosan-modified nanocarriers by a mere close contact of nanocarriers and the materials to be delivered in the absence of further treatment.

In a preferred embodiment, drugs are encapsulated inside chitosan-modified nanocarriers in an aqueous dispersion phase without using an organic dispersion phase.

In a preferred embodiment, the encapsulation is carried out at 0-20° C., more preferably 4-10° C., most preferably 4-6° C.

The aforementioned spontaneous encapsulation in aqueous solution can remarkably increase the stability of therapeutic agents, particularly protein drugs. Encapsulation efficiency is as high as 90% or higher in the spontaneous encapsulation inside the chitosan-modified nanocarriers herein. Moreover, a process of the present invention neither uses organic solvents during the drug loading step nor necessitates high-speed homogenization or ultrasonification generally carried out in the conventional process, thereby enabling to ensure the stability of therapeutic agents by avoiding denaturation or aggregation of the agents.

In a preferred embodiment, a targeting ligand is bound on the surface of chitosan-modified nanocarriers herein. Examples of a targeting ligand herein include without limitation a hormone, an antibody, a cell-adhesion molecules, a saccharide and a neurotransmitter.

In another aspect of this invention, there is provided a delivery method of a cargo comprising a step of contacting the chitosan-modified nanocarrier comprising the cargo material to a subject.

In another aspect of this invention, there is provided a process of preparing chitosan-modified nanocarrier having a diameter which changes in accordance with changes in temperature, has enhanced skin permeability, cellular uptake or selective delivery to cancer tissue as compared with a bare nanocarrier to which chitosan has not been bound, which comprises the steps of:

(a) preparing a dispersion comprising a water-soluble biocompatible polymer with photo-crosslinkable functional group;

(b) preparing a dispersion comprising a water-soluble chitosan with photo-crosslinkable functional group;

(c) preparing a mixture by mixing the dispersion comprising biocompatible polymer and the dispersion comprising water-soluble chitosan;

(d) adding an initiator to the mixture; and

(e) preparing a chitosan-modified nanocarriers by crosslinking the polymer and chitosan by irradiating light onto the product of step (d).

Although any conventional initiators can be used in the present invention without limitation, a preferred type of initiator is a radical photoinitiator that produces reactive species under irradiation of UV or visible light. Examples of a photoinitiator herein include but are not limited to ethyl eosin, 2,2-dimethoxy-2-phenyl acetophenone, 2-methoxy-2-phenylacetophenone, 2-hydroxy-1-[4-(2-hydroxyethoxy)phenyl]-2-methyl-1-propanone (Irgacure 2959 or Darocur 2959), camphorquinone, acetophenone, acetophenone benzyl ketal, 1-hydroxycyclohexy phenyl ketone, 2,2-dimethoxy-2-phenylacetophenone, xanthone, fluorenone, benzaldehyde, fluorene, anthraquinone, triphenylamine, carbazole, 3-methylacetophenone, 4-chlorobenzophenone, 4,4′-dimethoxybenzophenone, 4,4′-diaminobenzophenone, benzoin propyl ether, benzoin ethylether, benzyl dimethyl ketal, 1-(4-isopropylphenyl)-2-hydroxy-2-methylpropan-1-one, 2-hydroxy-2-methyl-1-phenylpropan-1-one, thioxanthone, diethylthioxanthone, 2-isopropylthioxanthone, 2-chlorothiothioxanthone, 2-methyl-1-[4-(methylthio)phenyl]-2-morpholino-propan-1-one, 2,4,6-trimethylbenzoyl diphenylphosphine oxide and bis-(2,6-dimethoxybenzoyl)-2,4,4-trimethylpentylphosphine oxide.

Irgacure 2959, a photoinitiator used in the Example below, is known as almost non-cytotoxic (Kristi S. Anseth, et al., Cytocompatibility of UV and visible light photoinitiating systems on cultured NIH/3T3 fibroblasts in vitro. J. Biomater. Sci. Polymer Edn., 2000. 11(5): P. 439-457).

The nanocarrier is prepared by crosslinking the polymer and the chitosan via photo-crosslinkable functional group by irradiating visible or UV light in step (e). Preferably, UV light is used for crosslinking. According to one example of the present invention, a UV lamp for a thin layer chromatography can be used for irradiating UV light for its relatively lower price and better availability. This UV lamp is also appropriate for an initiator that is decomposed to generate radicals by the radiation of 365 nm UV (e.g., Irgacure 2959).

In a preferred embodiment, the steps (a)-(e) are carried out in an aqueous dispersion phase without using an organic dispersion phase, i.e. in a single phase. More specifically, nanocarriers can be prepared by irradiating light onto an aqueous dispersion comprising a biocompatible polymer and an initiator. Moreover, the synthesis of the present invention is carried out via a one-pot reaction. In this respect, a process of the present invention can be referred to as a “one-pot single-phase synthesis”.

According to the example, a process of the present invention overcomes the conventional problems such as complicated preparation steps and the use of organic solvent. In addition, a process of the present invention can ensure the stability of drugs or aggregation without necessitating high-speed homogenization or ultrasonification generally carried out in the conventional process.

In another aspect of this invention, there is provided a composition for transdermal delivery comprising the chitosan-modified nanocarrier.

In another aspect of this invention, there is provided a transdermal delivery method comprising a step of contacting the chitosan-modified nanocarrier comprising the cargo material to a subject's skin.

In another aspect of this invention, there is provided a composition for in vivo tumour or cancer imaging comprising the chitosan-modified nanocarrier.

In another aspect of this invention, there is provided a method for in vivo tumour or cancer imaging of a subject, which comprises the steps of:

(a) administering a diagnostically effective dose of the chitosan-modified nanocarrier comprising the cargo material to the subject; and

(b) acquiring visible images by scanning the subject.

In still another aspect of this invention, there is provided a composition for photothermal cancer therapy comprising the chitosan-modified nanocarrier.

In still another aspect of this invention, there is provided a method for photothermal cancer therapy comprising the step of administering a therapeutically effective dose of chitosan-modified nanocarrier comprising the cargo material to a subject.

Since the present composition comprises the chitosan-modified nanocarrier of this invention as active ingredients described above, the common descriptions between them are omitted in order to avoid undue redundancy leading to the complexity of this specification.

As confirmed by the Example below, the chitosan-modified nanocarrier of the present invention has enhanced skin permeability as compared with a bare nanocarrier to which chitosan has not been bound. In addition, chitosan-modified nanocarrier of the present invention can be used as a composition for in vivo tumour or cancer imaging and as a composition for photothermal therapy of tumour cells and cancer cells, since the cellular uptake by tumour cells and cancer cells is substantially improved.

The composition for transdermal delivery in the present invention is generally a pharmaceutical composition, and can be formulated with a pharmaceutically acceptable carrier.

The material delivered by chitosan-modified nanocarriers used in the composition for transdermal delivery of the present invention include but are not limited to, wrinkle-improving agent effective for skin or scalp, moisturizing agent, acne treatment agent, dark spot removing agent, skin elasticity-improving agent, hair growth stimulating agent, skin anti-aging agent or epidermal stem cell proliferating agent.

In a preferred embodiment of the present invention, a nanocarrier of the composition for transdermal delivery includes a high molecular weight protein, a peptide, a nucleic acid, a saccharide, a lipid, a nanoparticle, a compound or an inorganic compound.

The term “high molecular weight” used herein refers to a molecular weight size unable to permeate the skin (preferably human skin), preferably the high molecular weight material is higher than 500 Da. Generally, materials of molecular weight less than 500 Da is known to permeated the skin (Bos J D, et al., Exp. Dermatol 9: 165-169 (2000)).

As describe above, the nanocarrier of the present invention has enhanced skin permeability, therefore enabling the transdermal delivery of high molecular weight materials (e.g., protein drugs) which were considered as impossible.

The pharmaceutically acceptable carrier contained in the pharmaceutical composition of the present invention, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

The pharmaceutical composition according to the present invention may be transdermally administered.

A suitable dosage amount of the pharmaceutical composition of the present invention may vary depending on pharmaceutical formulation methods, administration methods, the patient's age, body weight, sex, pathogenic state, diet, administration time, administration route, an excretion rate and sensitivity for a used pharmaceutical composition, and physicians of ordinary skill in the art can determine an effective amount of the pharmaceutical composition for desired treatment. Generally, the pharmaceutical composition of the present invention may be administered with a daily dose of 0.001-100 mg/kg (body weight).

According to the conventional techniques known to those skilled in the art, the pharmaceutical composition according to the present invention may be formulated with pharmaceutically acceptable carrier and/or vehicle as described above, finally providing several forms a unit dose form and a multi-dose form. Non-limiting examples of the formulations include, but not limited to, a solution, a suspension or an emulsion in oil or aqueous medium, an extract, an elixir, a powder, a granule, a tablet and a capsule, and may further comprise a dispersion agent or a stabilizer.

The pharmaceutical composition for transdermal delivery transports the cargo material transdermally by contacting a subject's skin (preferably, a mammal, most preferably, human).

The composition for photothermal cancer therapy in the present invention uses the characteristics of increased cellular uptake of chitosan-modified nanocarrier by tumor cells or cancer cells.

The composition for photothermal cancer therapy according to the present invention may be formulated with pharmaceutically acceptable carrier and/or vehicle as described above in the composition for transdermal delivery.

The chitosan-modified nanocarrier used in the composition for photothermal cancer therapy may include photosensitizer or heat generating material, preferably metal particle. Non-limiting examples of the metal particle include, but not limited to a gold particle, silicon particle and magnetic particle (e.g., iron oxide nanoparticle, ferrite, magnetite or permalloy).

The composition for photothermal cancer therapy may generate heat preferably by electromagnetic radiation. For example, gold nanoparticles may effectively induce tumour or cancer cell apoptosis by generating heat after infrared laser irradiation. When using magnetic nanoparticles, heat is generated by high frequency magnetic field.

The composition for photothermal cancer therapy according to the present invention may be parenterally administered. When administered parenterally, it is preferably administered by intravenous, subcutaneous, intramuscular, intraperitoneal, intratumoural or intralesional injection. A suitable dosage amount of the composition of the present invention may vary depending on pharmaceutical formulation methods, administration methods, the patient's age, body weight, sex, pathogenic state, diet, administration time, administration route, an excretion rate and sensitivity for a used nanomaterial. Generally, the pharmaceutical composition of the present invention may be administered with a daily dose of 0.001-100 mg/kg (body weight).

According to the conventional techniques known to those skilled in the art, the pharmaceutical composition of the present invention may be formulated with pharmaceutically acceptable carrier and/or vehicle as described above, finally providing several forms including a unit dose form and a multi-dose form. Non-limiting examples of the formulations include, but not limited to, a solution, a suspension or an emulsion in oil or aqueous medium, an elixir, a powder, a granule, a tablet and a capsule, and may further comprise a dispersion agent or a stabilizer.

The composition for photothermal cancer therapy may effectively induce cancer cell apoptosis in various cancer diseases such as stomach, lung, breast, ovarian, liver, bronchogenic, nasopharyngeal, laryngeal, pancreatic, bladder, colon, cervical, brain, prostatic, bone, skin, thymus, hyperthymus and ureteral carcinoma.

According to another aspect of the present invention, the present invention provides a composition for in vivo tumor or cancer imaging comprising the chitosan-modified nanocarrier as described above.

As proved by the example below, the chitosan-modified nanocarrier of the present invention may be used as an in vivo tumor or cancer imaging agent since the cellular uptake by tumour cells and cancer cells is significantly high.

In this case, the chitosan-modified nanocarrier of the present invention includes a compatible contrast agent or an imaging agent.

For example, compatible fluorescent material may be encapsulated inside the chitosan-modified nanocarrier or bound to the surface of the chitosan-modified nanocarrier when optical fluorescence is used for in vivo tumour or cancer imaging.

When using MRI as an in vivo tumour or cancer imaging method, particles generating paramagnetic, superparamagnetic or proton density may be included in the chitosan-modified nanocarrier for compatible T1 and T2 contrast. Examples of contrast agent include, Gd(III), Mn(II), Cu(II), Cr(III), Fe(II), Fe(III), Co(II), Er(II), Ni(II), Eu(III), Dy(III), pure iron, magnetic iron oxide (e.g., magnetite, Fe₃O₄), γ—Fe₂O₃, mangan ferrite, cobalt ferrite, nickel ferrite and perfluorocarbon.

When the imaging composition of the present invention is used for single photon emission computed tomography (SPET) or positron emission topography (PET) imaging, the chitosan-modified nanocarrier may include positron emitting isotope, for example, ¹¹C, ¹³O, ¹⁴O, ¹⁵O, ¹²N, ¹³N, ¹⁵F, ¹⁷F, ¹⁸F, ³²Cl, ³³Cl, ³⁴Cl, ⁴³Sc, ⁴⁴Sc, ⁴⁵Tl, ⁵¹Mn, ⁵²Mn, ⁵²Fe, ⁵³Fe, ⁵⁵Co, ⁵⁶Co, ⁶¹Cu, ⁶²Cu, ⁶³Zn, ⁶⁴Cu, ⁶⁵Zn, ⁶⁶Ga, ⁶⁶Ge, ⁶⁷Ge, ⁶⁸Ga, ⁶⁹Ge, ⁶⁹As, ⁷⁰As, ⁷⁰Se, ⁷¹Se, ⁷¹As, ⁷²As, ⁷³Se, ⁷⁴Kr, ⁷⁴Br, ⁷⁵Br, ⁷⁶Br, ⁷⁷Br, ⁷⁷Kr, ⁷⁸Br, ⁷⁸Rb, ⁷⁹Rb, ⁸¹Rb, ⁸²Rb, ⁸⁴Rb, ⁸⁴Zr, ⁸⁵Y, ⁸⁶Y, ⁸⁷Y, ⁸⁸Y, ⁸⁹Zr, ⁹²Tc, ⁹³Tc, ⁹⁴Tc, ⁹⁵Tc, ⁹⁵Ru, ⁹⁵Rh, ⁹⁶Rh, ⁹⁷Rh, ⁹⁸Rh, ⁹⁹Rh, ¹⁰⁰Rh, ¹⁰¹Ag, ¹⁰²Rh, ¹⁰³Ag, ¹⁰⁴Ag, ¹⁰⁵Ag, ¹⁰⁶Ag, ¹⁰⁸In, ¹⁰⁹In, ¹¹⁰In, ¹¹⁵Sb, ¹¹⁶Sb, ¹¹⁷Sb, ¹¹⁵Te, ¹¹⁶Te, ¹¹⁷Te, ¹¹⁷I, ¹¹⁸I, ¹¹⁸Xe, ¹¹⁹Xe, ¹¹⁹I, ¹¹⁹Te, ¹²⁰I, ¹²⁰Xe, ¹²¹Xe, ¹²¹I, ¹²²I, ¹²³Xe, ¹²⁴I, ¹²⁶I, ¹²⁸I, ¹²⁹La, ¹³⁰La, ¹³¹La, ¹³²La, ¹³³La, ¹³⁵La, ¹³⁶La, ¹⁴⁰Sm, ¹⁴¹Sm, ¹⁴²Sm, ¹⁴⁴Gd, ¹⁴⁵Gd, ¹⁴⁵Eu, ¹⁴⁶Gd, ¹⁴⁶Eu, ¹⁴⁷Eu, ¹⁴⁸Eu, ¹⁵⁰Eu, ¹⁹⁰Au, ¹⁹¹Au, ¹⁹²Au, ¹⁹³Au, ¹⁹³Tl, ¹⁹⁴Tl, ¹⁹⁴Au, ¹⁹⁵Tl, ¹⁹⁶Tl, ¹⁹⁷Tl, ¹⁹⁸Tl, ²⁰⁰Tl, ²⁰⁰Bi, ²⁰²Bi, ²⁰³Bi, ²⁰⁵Bi, ²⁰⁶Bi, or derivatives thereof.

When the imaging composition of the present invention is used for computed tomography (CT) imaging, the chitosan-modified nanocarrier may include CT contrast agents such as an iodide or a gold particle.

### Advantageous Effects

The features and advantages of the present invention will be summarized as follows:

(a) Chitosan-modified nanocarrier of the present invention showed significantly high level of improvement in skin permeability compared with a bare nanocarrier that has no chitosan, thus exhibiting excellent efficacy.

(b) Chitosan-modified nanocarrier of the present invention can be advantageous in the imaging and photothermal therapy of tumour cells and cancer cells, since the cellular uptake by tumour cells and cancer cells is substantially improved.

(c) Nanocarrier of the present invention is temperature-sensitive, and their average diameter and pore size reversibly change in response to temperature change.

(d) Chitosan-modified nanocarrier can be prepared via a one-pot single-phase synthesis.

(e) A material to be delivered can be spontaneously encapsulated inside nanocarrier.

(f) Nanocarrier of the present invention can be used as a sustained-release drug delivery system because the pores of nanocarrier herein decreases at a human body temperature

(g) A process of the present invention overcomes the conventional problems such as the use of organic solvent, complicated preparation steps, a relatively high manufacture cost and a low loading efficiency.

(h) And, a process of the present invention can ensure the stability of drugs without necessitating high-speed homogenization or ultrasonification generally carried out in the conventional process.

### DESCRIPTION OF DRAWINGS

FIG. 1a is a diagram illustrating the production of glycidyl metaacrylated chitooligosaccharide: GMA-COS for producing chitosan-modified nanocarrier.

FIG. 1b is a ¹H-NMR spectroscopy result confirming the synthesis of GMA-COS of FIG. 1a.

FIG. 2 is a diagram illustrating the production of chitosan-modified nanocarrier of the present invention.

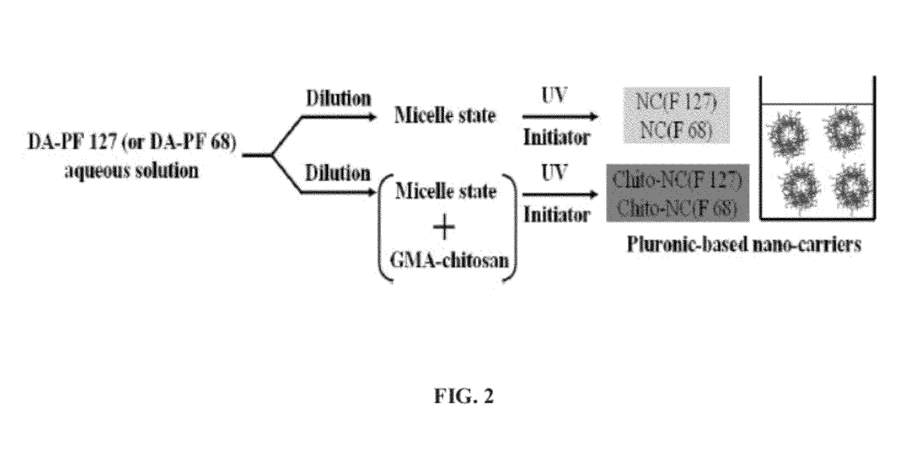

FIG. 3 is graphs represents the size and zeta potential of chitosan-modified nanocarrier.

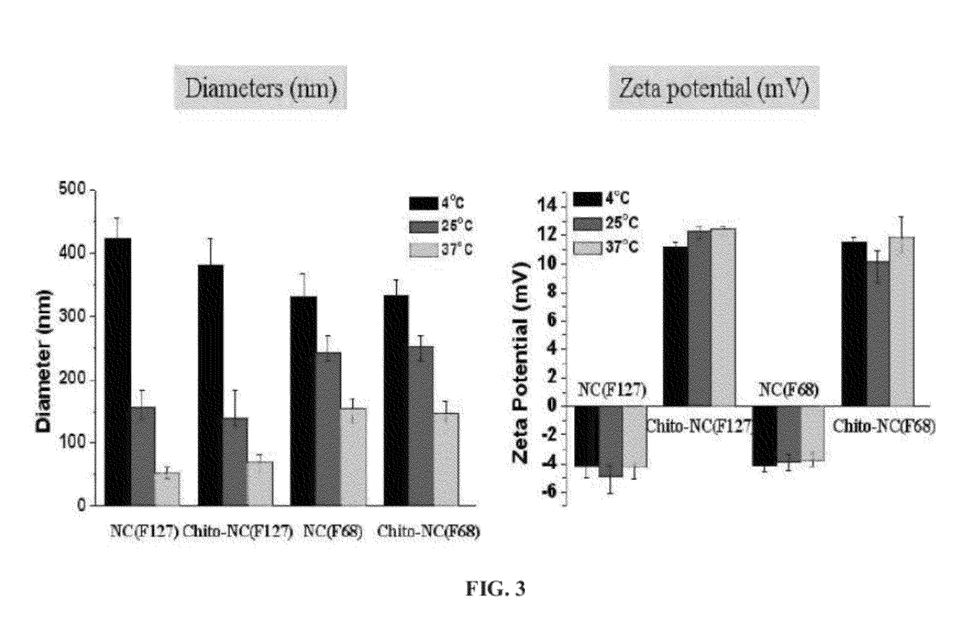

FIG. 4a is a diagram illustrating the static Franz-type diffusion cell used for measuring skin permeability.

FIG. 4b is a graph representing the in vitro skin permeability of FITC-BSA loaded chitosan-modified nanocarrier.

FIG. 4c is fluorescence microscope images showing the distribution of FITC-BSA loaded chitosan-modified nanocarrier after skin permeation.

FIG. 4d is a graph representing in vitro skin permeability of Cy5.5 loaded chitosan-modified nanocarrier.

FIG. 5a is a flow cytometery result showing the in vitro cell uptake of chitosan-modified nanocarrier in SCC7 cell line.

FIG. 5b is in vivo NIR fluorescence images showing the real-time tumour targeting of chitosan-modified nanocarrier (loaded with Cy5.5) using SCC7 cell transplanted tumour mouse models.

FIG. 5c is graphs showing the quantification of in vivo tumour targeting of chitosan-modified nanocarrier (loaded with Cy5.5) in a time dependent manner.

FIG. 5d is a graph representing the quantification of chitosan-modified nanocarrier (loaded with Cy5.5) distributed in organs and accumulated in tumour.

FIG. 5e is ex vivo NIR fluorescence images of organ and tumour confirming the chitosan-modified nanocarrier (loaded with Cy5.5) distributed in organs and accumulated in tumour.

FIG. 6a is TEM image and NIR spectrum profile of gold nanoparticle of chitosan-modified nanocarrier which is used for bio imaging and in vivo imaging.

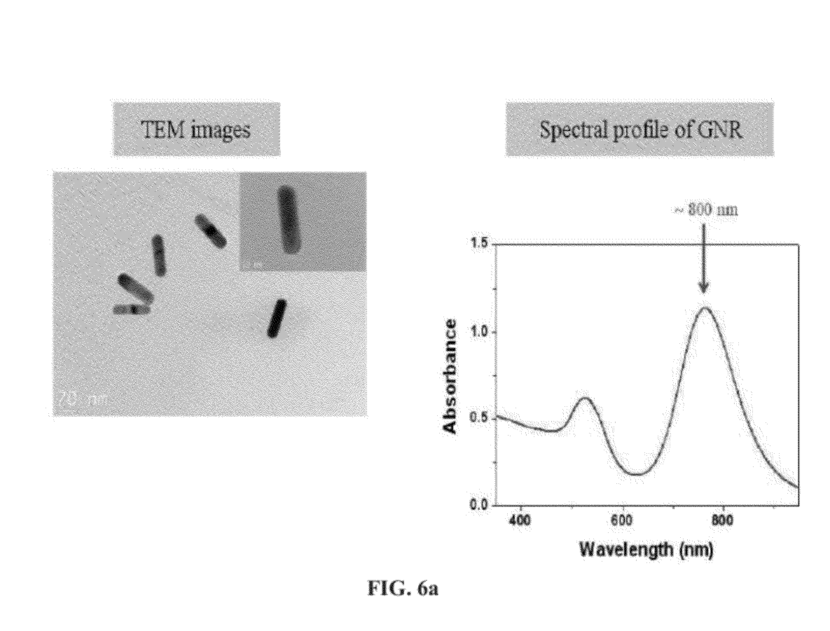

FIG. 6b is graphs representing the stability analysis of gold nanoparticle loaded chitosan-modified nanocarrier.

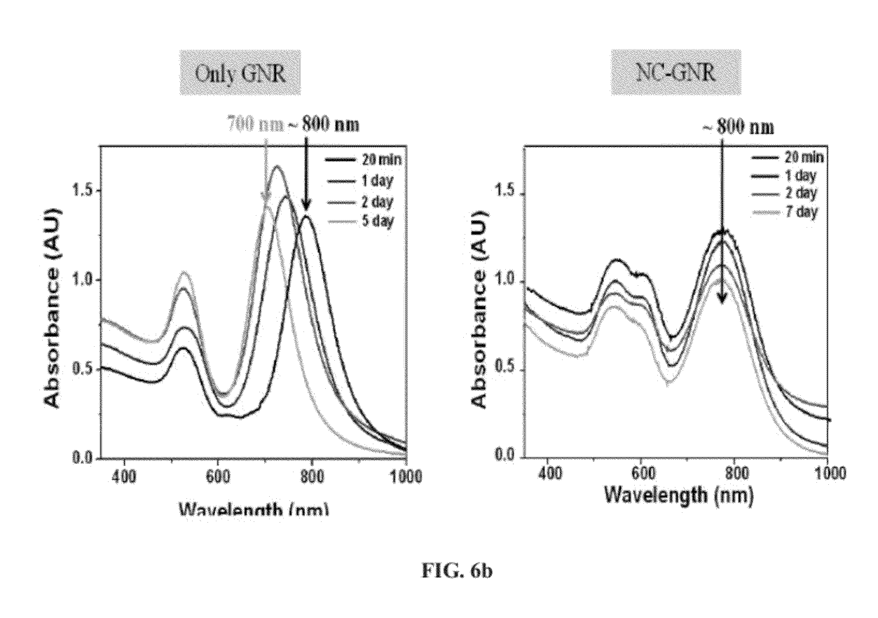

FIG. 6c is images showing the cellular uptake of gold nonorod and GNR loaded chitosan-modified nanocarrier.

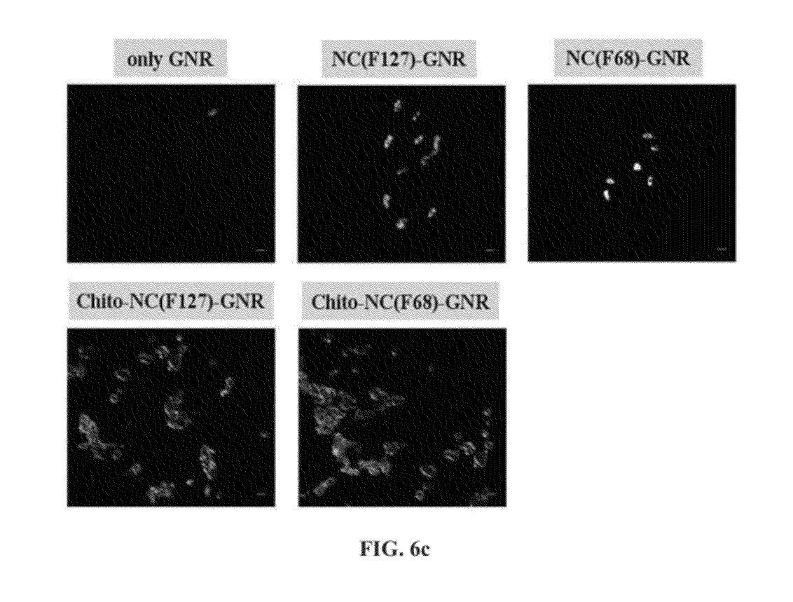

FIG. 6d is images showing the in vitro photothermal therapy using chitosan-modified nanocarrier loaded with GNR. The cw laser (a diode continuous-wave laser) was used at 41.5 W/cm².

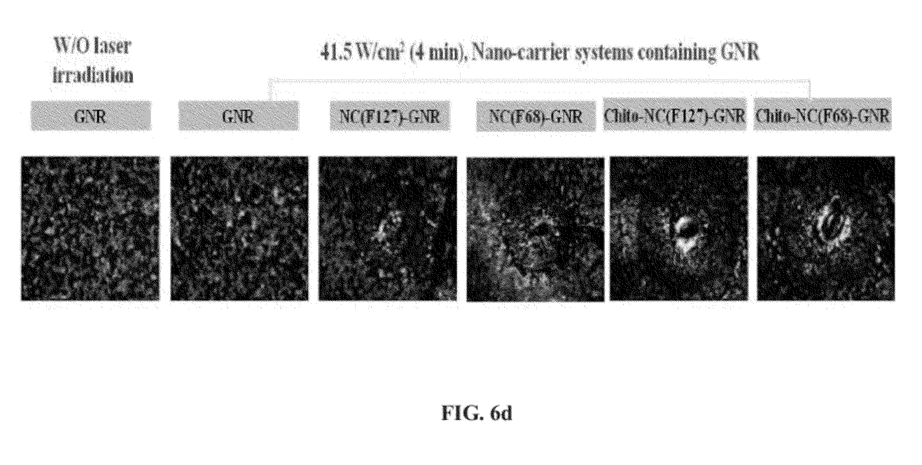

FIG. 6e is images showing the in vitro photothermal therapy using chitosan-modified nanocarrier loaded with GNR. The cw laser (a diode continuous-wave laser) was used at 26.4 W/cm².a

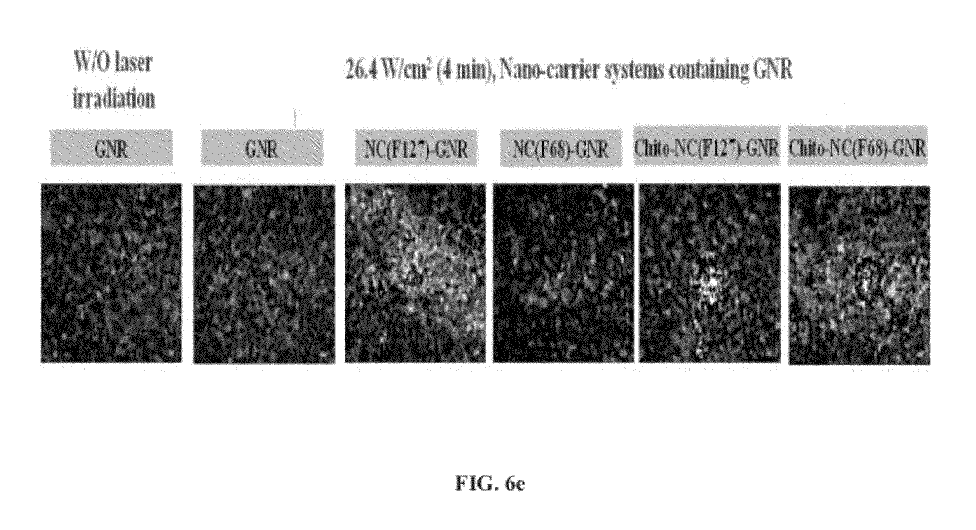

FIG. 7a is TEM images showing the absorption spectra of GNR, GNR loaded in nanocarrier and GNR loaded in chitosan-modified nanocarrier.

FIG. 7b is graphs representing the size (diameter) and zeta potential of nanocarrier and GNR loaded nanocarrier.

FIG. 8 is a graph representing the cumulative leakage of GNR from nanocarrier and chitosan-conjugated nanocarrier into PBS leakage

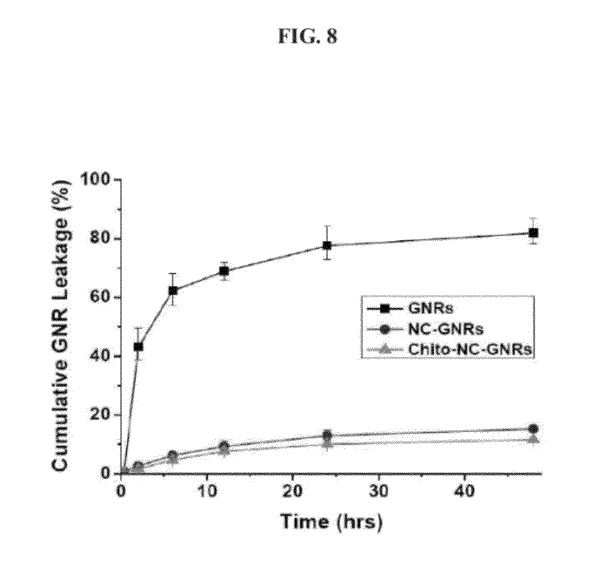

FIG. 9 is light scattering images of cells observed by using a dark-field microscope after applying GNR, GNR loaded nanocarrier and chitosan-conjugated nanocarrier.

FIG. 10 is fluorescence images showing cytotoxicity after selective NIR photothermal therapy on SCC7 cancer cells (panel a) and NIH/3T3 fibroblast cells (panel b) with GNRs or GNR-loaded nanocarriers by laser irradiation at 780 nm with two different power densities (41.5 and 26.4 W/cm²).

FIG. 11 is sliver staining images of observing the absorption of the GNR, GNR-loaded nanocarrier and chitosan-conjugated nanocarrier in tumour cells and liver cell after intravenous injection.

FIG. 12a is graph showing the changes in tumour volume after NIR laser irradiation at 24 hrs after the i.v. injection of the GNRs, GNR-loaded nanocarriers and chitosan-conjugated nanocarrier.

FIG. 12b is images of mouse tumour representing the changes in tumour sizes after NIR laser irradiation at 24 hrs after the i.v. injection of the GNRs, GNR-loaded nanocarriers and chitosan-conjugated nanocarrier.

FIG. 12c is graph showing the changes in tumour volume after one or two NIR laser irradiations at 24 and 48 hrs after the i.v. injection of the GNRs, GNR-loaded nanocarriers and chitosan-conjugated nanocarrier.

FIG. 12d is images of mouse tumour representing the changes in tumour sizes after one or two NIR laser irradiations at 24 and 48 hrs after the i.v. injection of the GNRs, GNR-loaded nanocarriers and chitosan-conjugated nanocarrier.

FIG. 13 is images showing the accumulation of pluronic-based nanocarriers and chitosan-conjugated nanocarriers in tumour cells at 72 hrs after the i.v. injection in nude mouse, respectively (A: whole body image of mouse, B: enlarged image of tumour site).

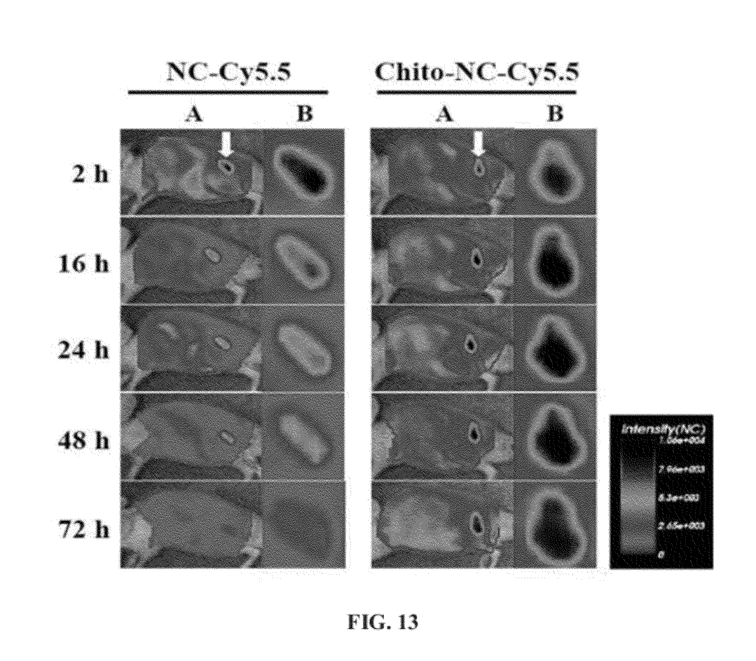

FIG. 14 is a schematic description of preparing pluronic-based nanocarriers and uptake of the GNR into nanocarrier.

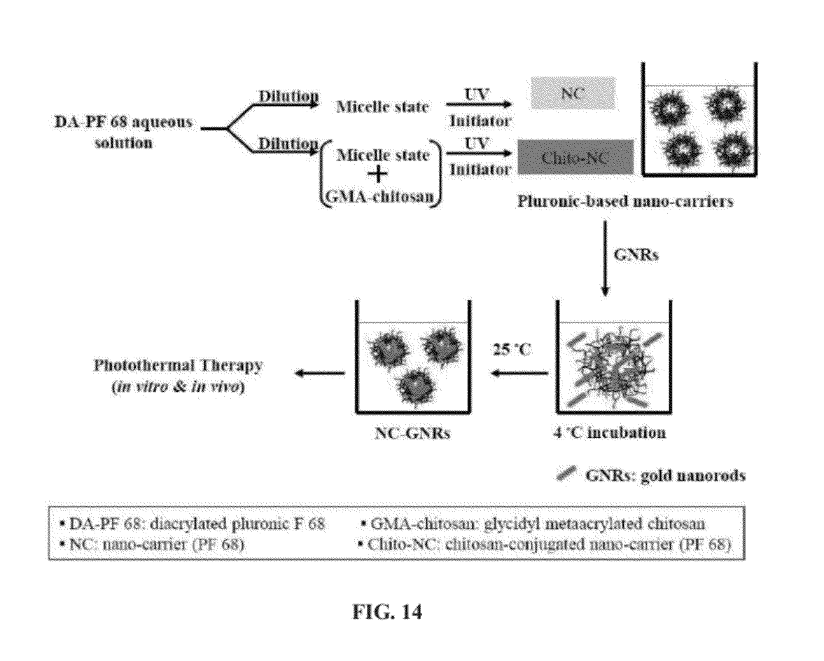

FIG. 15 is a graph showing the difference in cell viability after cellular uptake of different concentrations of GNRs, GNR-loaded nanocarriers and chitosan-conjugated nanocarriers in tumour and fibroblast cells.

FIG. 16 is a graph showing the amount of nanocarriers taken up by the SCCy tumour cells (a) and NIH/3T3 fibroblast cells (b) after incubating for 2, 12 and 24 hrs.

### BEST MODE

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art.

### EXAMPLES

### Example 1

### Preparation of GMA-Chitooligosaccharide (GMA-COS)

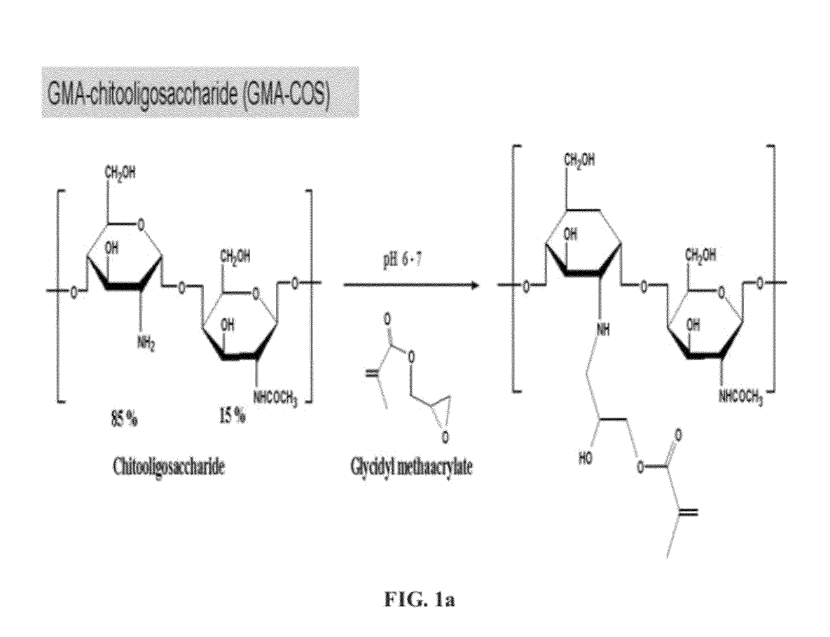

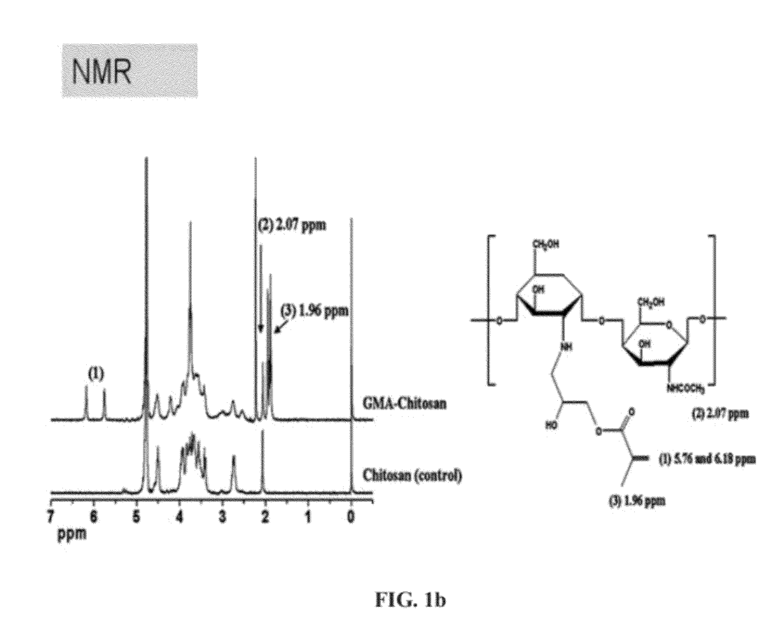

Glycidyl metaacrylated chitooligosaccharide: GMA-COS was prepared by using chitooligosaccharide and glycidyl metaacrylate and according to the method described in FIG. 1a. FIG. 1b is the ¹H-NMR spectroscopy (JNM-LA300WB FT-NMR Spectrometer, JEOL, Japan) analysis data of final product, GMA-COS, indicted that GMA-COS was successfully prepared.

### Example 2

### Preparation of Chitosan-Modified Nana-Carrier

Two types of Pluronic-based nanocarriers (NC) including a bare form (NC(PF 68)) and a chitosan-conjugated form (Chito-NC(PF 68)) were prepared by photo-polymerizing diacrylated Pluronic (DA-Pluronic) and acrylated chitosan, as previously reported by the present inventors (32,33). Briefly, for preparation of the bare form, dilute aqueous solution (2 mL) of diacrylated Pluronic (0.5 wt %) was gently mixed with a photoinitiator [0.05 wt % Irgacure 2959, 4-(2-hydroxyethoxy) phenyl-(2-hydroxy-2-propyl) ketone, Ciba Specialty Chemicals Inc], followed by UV irradiation for 15 min with 1.3 mW/cm² intensity using an unfiltered UV lamp (VL-4.LC, 8W, Vilber Lourmat, France). In the case of chitosan-conjugated form, a water soluble glycidyl-methaacrylate (GMA)-conjugated chitosan (2.8 mg, 0.2 μmol) was dissolved in de-ionized water and added into a DA-Pluronic solution to make 0.5 wt % of DA-Pluronic. This mixture was photo-polymerized at the same condition used for the bare form to allow incorporation of vinyl groups of GMA-conjugated chitosan into the crosslinked nanocarrier. To remove un-reacted substances, the whole solution was dialyzed using a dialysis bag (cellulose ester, MWCO of 300 kDa) first in 0.1M NaCl and followed by a second dialysis in de-ionized water. Next, the sizes and the surface charges of both kinds of Pluronic-based nanocarriers were analyzed using an electrophoretic light scattering spectrophotometer (ELS-Z2, Otsuka Electronics Co., Japan) equipped with a laser diode light source (638 nm) and a photo multiplier tube detector (165° scattering angle). In the case of chitosan-conjugated type, the amount of chitosan incorporation determined by Ninhydrin assay was found to be 16 wt %.

### Example 3

### Analysis of Skin Permeation of Chitosan-Modified Nanocarrier (Using FITC-BSA)

The model protein FITC-BSA (Fluorescein isothiocyanate-labeled bovine serum albumin) was loaded into the chitosan-modified nanocarrier prepared form the Example. The model protein, FITC-BSA was added to the chitosan-modified nanocarrier solution and incubated at 4° C. for over 12 h to induce spontaneous loading of the protein into the nanocarriers. Unloaded model proteins were removed by spin filtration at room temperature. The encapsulation efficiency and the loading amount of the protein inside the nanocarriers were determined after spin filtration at 14,000 rpm for 10 min at room temperature and were calculated by a method reported by F. Q. Li. et al., Int. J. Pharm., 2008, 349, 247.

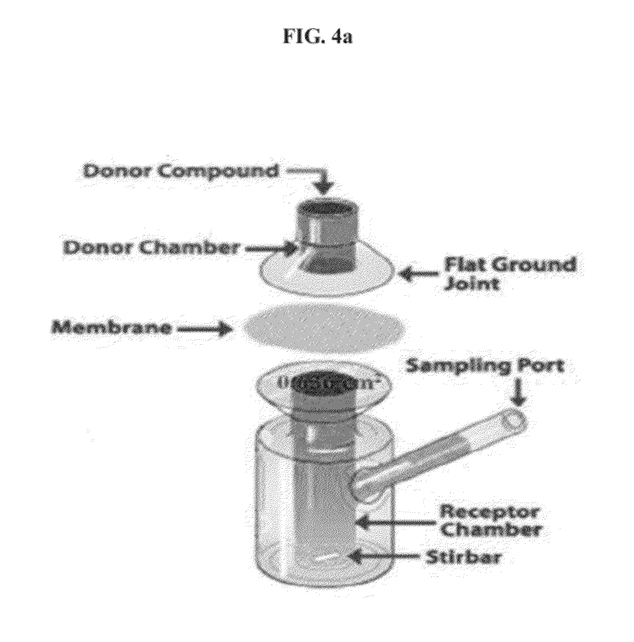

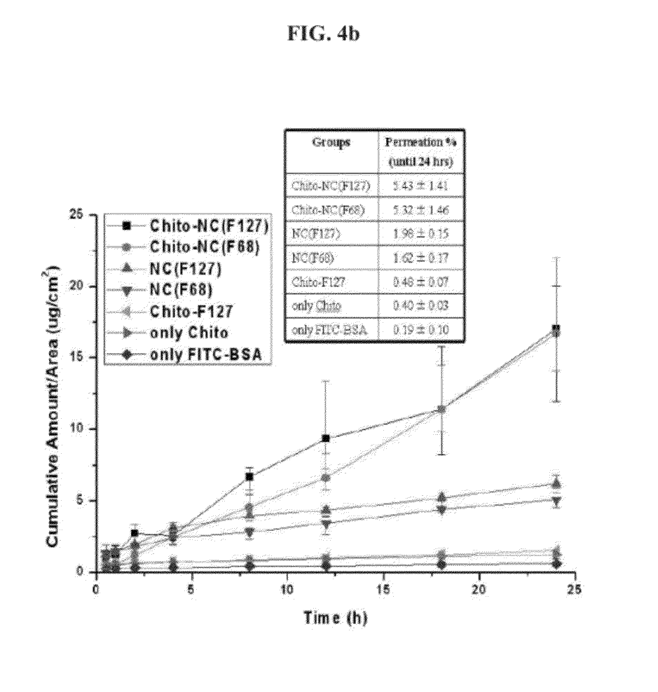

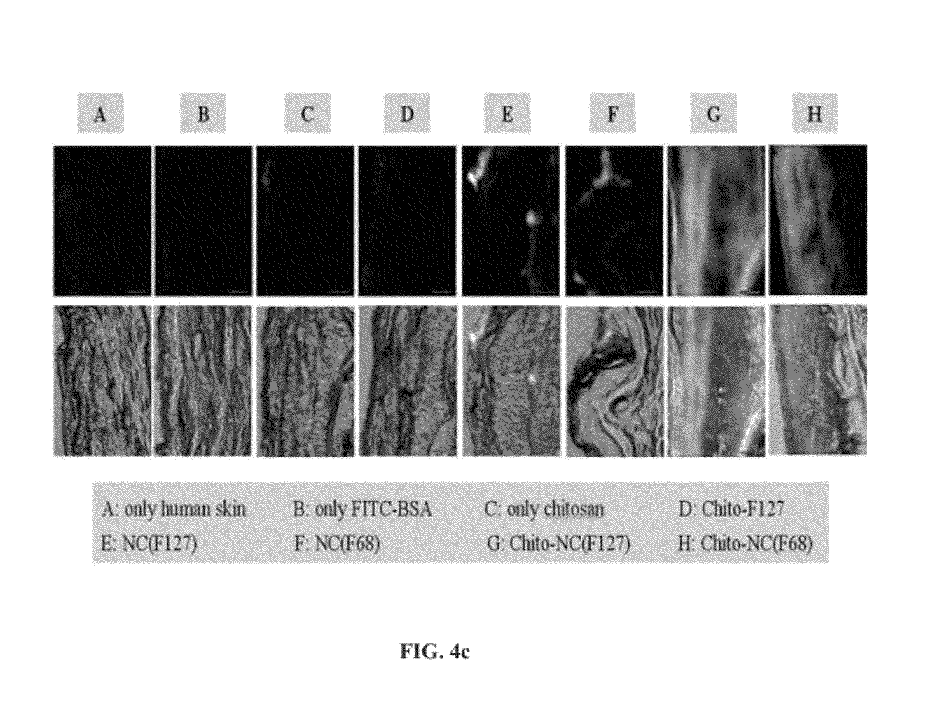

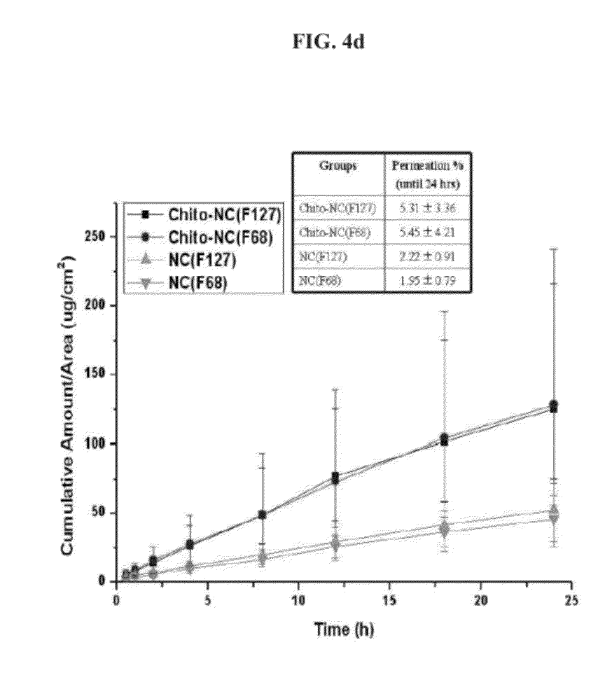

The skin penetration of FITC-BSA loaded nanocarrier was measured by using the Franz-type diffusion cell (see FIG. 4a). The experimental group is as follows; only FITC-BSA (200 μg), NC (F127)+FITC-BSA, NC (F68)+FITC-BSA, Chito-NC (F127)+FITC-BSA, Chito-NC (F68)+FITC-BSA, Only chitosan and Chito-F127. The experimental condition is as follows; Donor chamber: 1-5 groups in DIW (200 μA); Membrane: Epidermis & dermis (Human cadaver skin, M/58. back and thigh) (from HANS Biomed); Receptor chamber: PBS (pH 7.4) (5 ml); 31, 7° C., 600 rpm, time point (0.5, 1, 2, 4, 8, 12, 18 and 24 hrs); Sampling: 500 μl at given time. The fluorescence intensity was measured by spectrofluoro photometer and the fluorescence images were collected by fluorescence microscopy.

As represented in FIG. 4b, the chitosan-modified nanocarrier in the present invention showed more efficient skin penetration when compared with chitosan un-conjugated nanocarriers [NC(F127) and NC(F68)]. In addition, the chitosan-modified nanocarrier in the present invention showed more efficient skin penetration when compared with Chito-F127, in which the chitosan is conjugated to the Pluronic complex, but lacks photo-crosslinking. FIG. 4c is fluorescence image results after treating the skin with FITC-BSA loaded chitosan-conjugated nanocarrier. Similarly, the chitosan-modified nanocarrier showed more efficient skin penetration when compared with chitosan un-conjugated nanocarriers [NC(F 127) and NC(F68)].

### Example 4

### Analysis of Skin Permeation of Chitosan-Modified Nanocarrier (Using Cy5.5)

Similar to Example 3, the skin penetration of fluorescence material, Cy5.5 conjugated nanocarrier was measured. As shown in FIG. 4d, the chitosan-modified nanocarrier showed more efficient skin penetration when compared with chitosan un-conjugated nanocarriers [NC(F 127) and NC(F68)].

### Example 5

### In Vivo Imaging Using Chitosan-Modified Nanocarrier

The possible application of fluorescence material Cy5.5 conjugated chitosan-modified nanocarrier was determined.

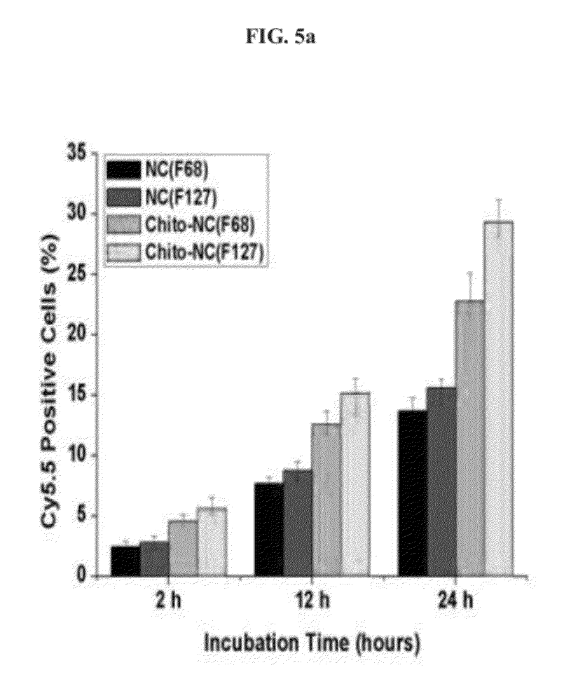

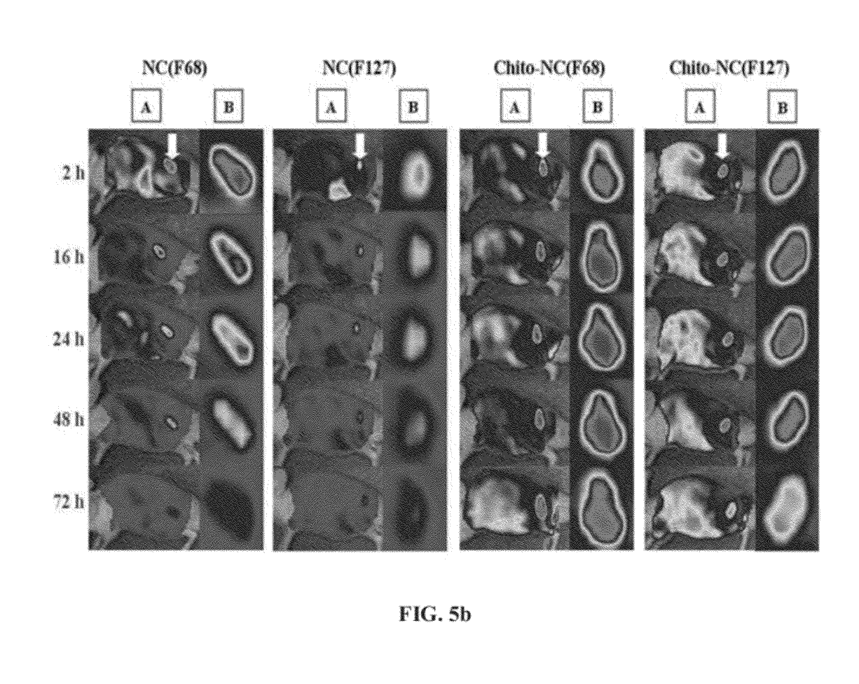

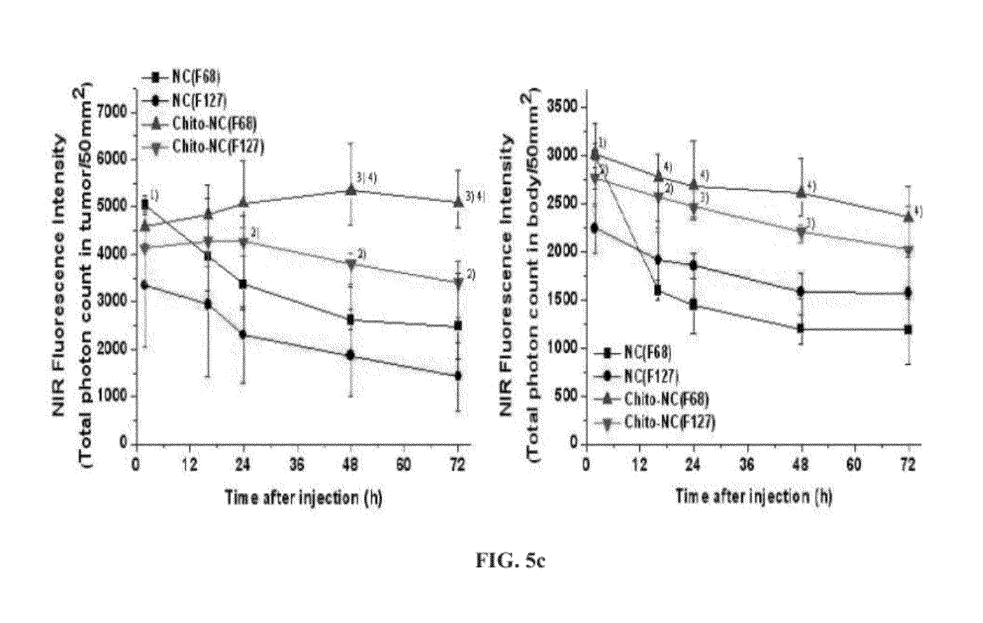

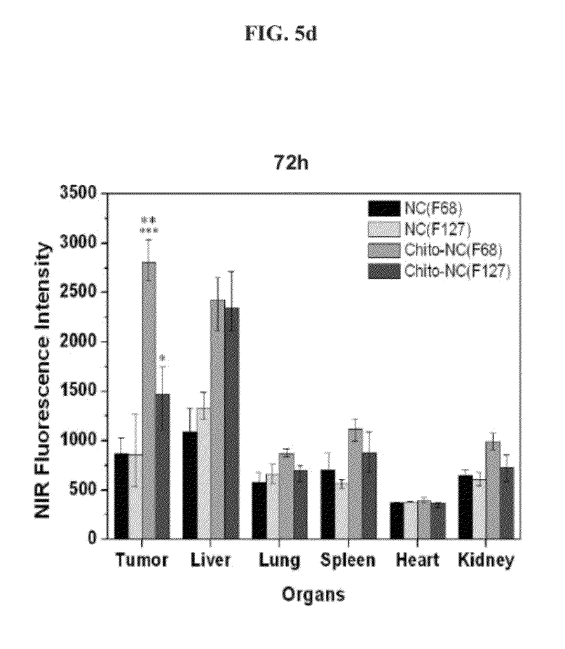

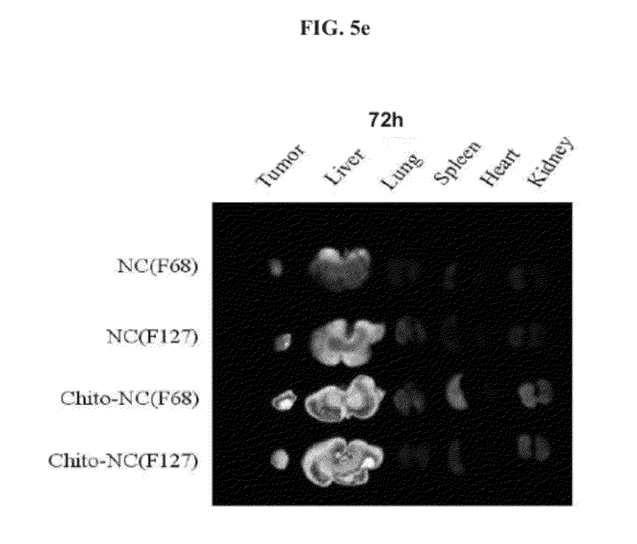

First, in vitro cellar uptake was examined by culturing the squamous cell carcinoma (SCC7). The extents of the cellular uptake of chitosan-modified nanocarrier were much higher than the bare nanocarriers. The increase of cellular uptake from in vitro experiments correlated with the in vivo data showing increased fluorescence intensities in tumours of SCC7 tumour bearing mouse model (FIGS. 5b, 5c and 5d). As shown in FIG. 5b, the time-dependent excretion profile and tumour accumulation was clearly presented by monitoring the NIR fluorescence intensity. In the case of bare nanocarrier [NC(F127) and NC(F68)], the fluorescent signals of the tumour site decreased rapidly, within 16 hr post injection. However, the high fluorescence intensity of the chitosan-modified nanocarrier remained until 72 hrs in the tumour site.

As indicated by the ex vivo NIR fluorescence image 73 hr post injection (FIGS. 5d and 5e), when the organ distribution (liver, lung, kidney, spleen and heart) and tumour accumulation was examined, the chitosan-modified nanocarrier showed higher fluorescence intensity in the tumour site compared to the bare nanocarrier. The result shows that the chitosan-modified nanocarrier has prolonged blood circulation time and increased tumour accumulating ability than that of the bare nano-carriers.

### Example 6

### In Vitro Photothermal Cancer Therapy Using Chitosan-Modified Nanocarrier

The result in Example 5 showing that the chitosan-modified nanocarrier has prolonged blood circulation time and increased tumour accumulating ability suggests a possible use as photothermal cancer therapeutic agent. The therapeutic application of chitosan-modified nanocarrier was examined.

First, the GNRs (GNRs) were synthesized by a seed-mediated growth method in aqueous CTAB solution (36). Gold seed was prepared by adding HAuCl₄ (0.5 mM, 5 mL, Kojima chemical Co. LTD (Ksashiwabara, Japan)) into CTAB solution (0.2 M, 5 mL) and mixing vigorously. And then, ice cold NaBH₄ (0.01M, 600 μL, Sigma-Aldrich Corp, USA) was added under vigorous stirring to produce brownish-yellow solution. The solution is incubated for 1-3 hr at room temperature to use as the seed solution for synthesizing the GNRs. To synthesize GNRs, a growth solution was prepared by adding HAuCl₄ (0.5 mM, 5 mL) into CTAB solution (0.2 M, 5 mL, Sigma-Aldrich Corp, USA) under vigorous stirring. 400 μL of 4 mM AgNO₃ (silver nitrate) and 70 μL of 0.0788 M ascorbic acid (Sigma-Aldrich Corp, USA) is added and then mixed gently. During this process, the yellow color of the growth solution becomes colorless. Subsequently, 12 μL of gold seed solution was added to the growth solution, and the mixture was stirred vigorously. The product solution was kept in a 37° C. shaking rocker at 100 rpm for 3 hrs. The color of the GNR changes from colorless to reddish-brown. To remove excess CTAB, the GNR solution was purified by centrifugation at 11 000 rpm for 10 min for at least five-fold purification and re-dispersed in de-ionized water. Finally, the absorbance of GNRs was measured using an UV-V is spectrometer (Agilent 8453, Santa Clara, Calif., USA) and their size distributions were measured using a high resolution transmission electron microscope (TEM; JEM-2100 LAB6, JEOL, Japan).

However, the GNR loaded Pluronic-based nanocarrier was prepared and characterized as follows. To load the GNR into the chitosan-modified nanocarrier, GNR solution (50 mg/100 μL) was added to the powdered nanocarrier (750 mg), followed by incubation at 4° C. for over 12 hr to induce spontaneous loading of the GNR into the nanocarriers. The encapsulation efficiency (above 90%) and the loading amount of the protein inside the nanocarriers were determined after removing the gold nanocarrier by spin filtration at 14,000 rpm for 10 min at room temperature and then calculated (44). The absorption spectrum of the GNRs and GNR loaded nanocarriers were measured in the visible-NIR wavelength range using an UV-spectrophotometer. The morphology of the GNR and GNR loaded nanocarrier was negatively stained with 2% (w/v) phospho-tungstic acid solution (Sigma-Aldrich Corp, USA) and observed under TEM. The sizes and the surface charges (zeta potential) of GNRs and GNR loaded nanocarriers in 37° C. de-ionized water were analyzed using an electrophoretic light scattering spectrophotometer (ELS-Z2, Otsuka Electronics Co., Japan). All measurements were performed in triplicates.

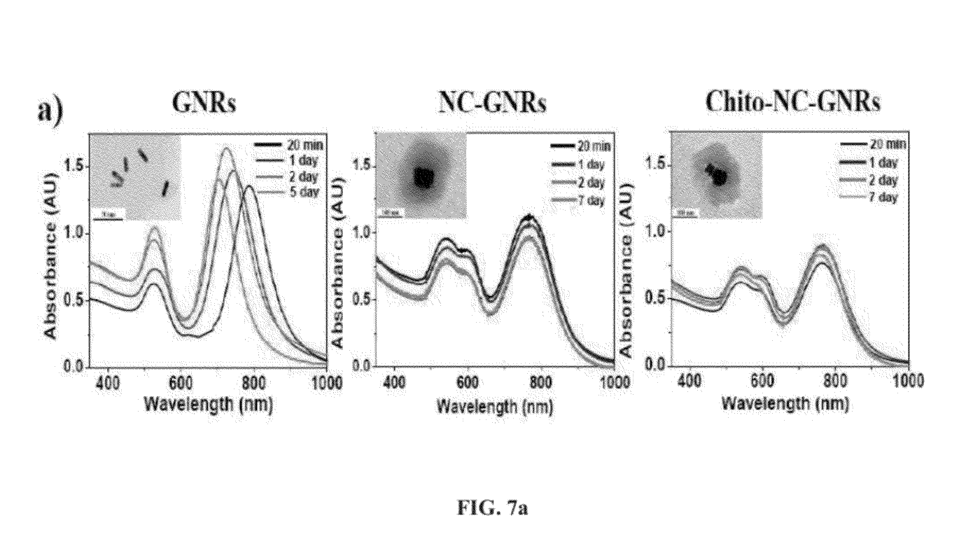

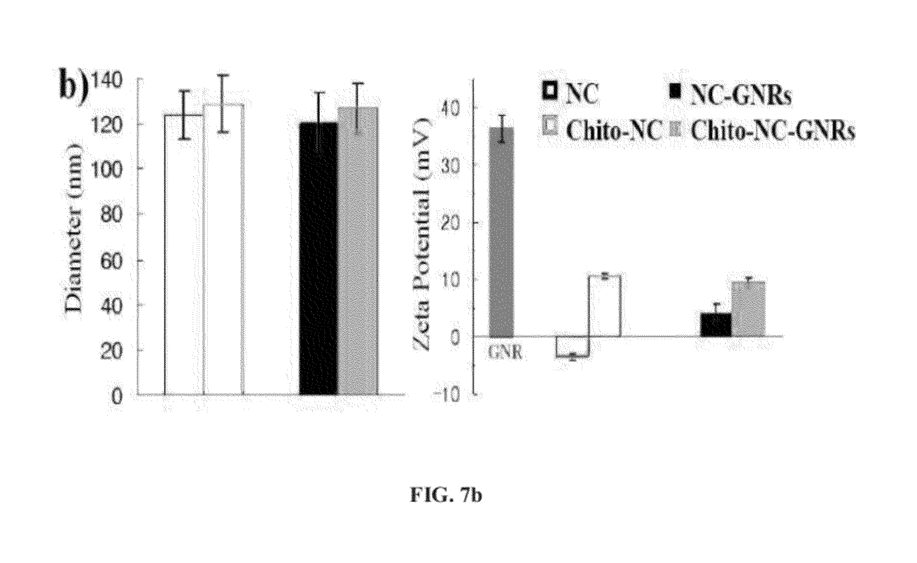

The morphology of the GNR and GNR loaded nanocarrier was negatively stained with phospho-tungstic acid solution and the images were observed under TEM (the inserted images in FIG. 7a). Adequate amounts of GNRs were encapsulated without changing the spherical shape of the nanocarriers. The hydrodynamic diameters and zeta potential were not affected by the encapsulation of GNRs at 37° C. As shown in FIG. 7g, nanocarrier alone and the GNR loaded nanocarrier have similar average sizes. The zeta potential of GNRs stabilized in CTAB solution shows high positive charge on the surface (+36.5±2.4 mV), however GNR loaded nanocarriers have similar surface charges as the zeta potential of nanocarriers, which is effective for encapsulating the GNRs into the nanocarrier.

The optical stability of the GNRs and GNR loaded nanocarriers dispersed in the aqueous solution was analyzed at different time points (FIG. 7a). The reshaping of GNRs in the aqueous solution reshapes, and generating blue shift (short wavelength) spectrum has been published in our previous research (37, 38), therefore the use of GNRs in the aqueous solution is limited. However, nanocarriers loaded with GNRs did not show any change in their absorption spectrum even at day 7, which indicates that the GNR is encapsulated by the interaction between GNR and the nanocarrier, therefore preventing the unstable reshaping of GNRs (38).

The in vitro stability of GNR loaded nanocarrier was characterized. To study the optical stability of GNRs loaded in the nanocarrier, 1 mL of de-ionized water solution containing GNR (as a control) and GNR loaded nanocarrier was cultured for 1 week at 27° C. on a 100 rpm shaking rocker. The absorbance of solution was monitored and analyzed by using UV-Vis spectrum at 350 nm to 1000 nm range. In order to test whether the GNR was stably stored in the nanocarrier, the leakage of GNR was measured. GNR loaded nanocarrier solution (100 μL) was applied to the dialysis membrane (cellulose ester, MWCO of 300 kDa). The dialysis membrane was dialyzed in 5 mL of PBS containing 10% FBS (Gibco (Grand Island, N.Y., USA) and incubated in a 37° C. shaking rocker at 100 rpm. The released medium was exchanged at each time point to maintain optimal sink condition. The amount of GNRs leaked at each time point was analyzed by using UV-spectro photometer, and the concentration was quantified by calibration curve. The amount of GNR leaked at identical dialysis membrane set up condition was used as the control group.

Only 15% of GNR inside the nanocarrier leaked out when compared to nearly 80% in the control group, confirming that the nanocarrier can effectively capture the GNRs from inside.

The in vitro cytotoxicity of the GNR loaded nanocarrier was characterized. The cytotoxicity of GNR and GNR loaded nanocarrier was characterized using squamous cell carcinoma (SCC7) cells and NIH/3T3 fibroblast cell line. Cells were seeded in a 24-well tissue culture plate at a density of 5×10⁴ cells per well. The GNR or GNR loaded nanocarriers (contains 6.7 wt % of GNR) were added to the plate well in the range of 1-250 μg/mL (based on the GNR amount). Cells were then incubated with the culture medium containing nanocarriers for 24 h at 37° C. Next, the medium was replaced with 825 μL of fresh medium containing 10-time diluted WST-1 (Biovision Inc., Mountain View, USA) and cells were further incubated for 2 h at 37° C. The absorbance of the colored medium was measured at 450 nm, using a scanning multi-well spectrophotometer (FL600, Bio-Tek®, Vermont, USA). The cytotoxicity method from the previous research using SSC7 cells and Pluronic-based nanocarrier was used (33).

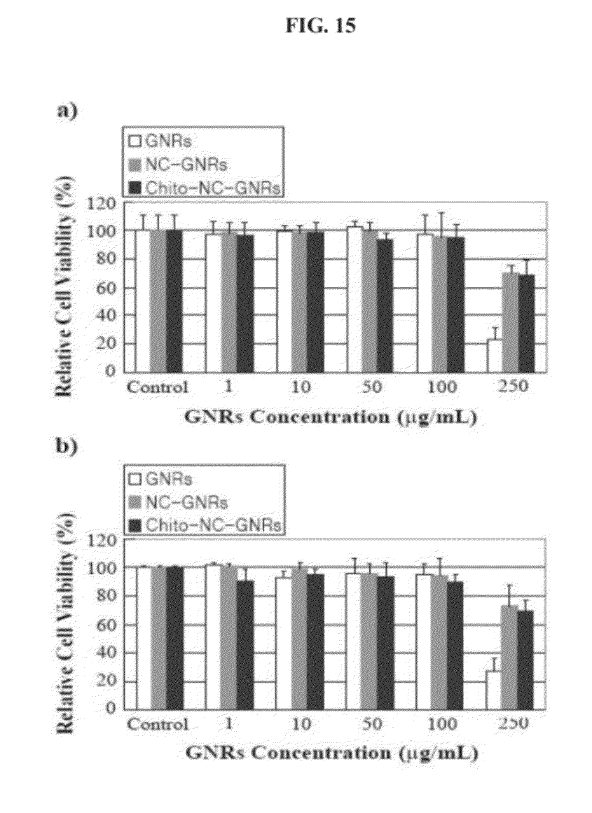

The same protocol was used for analyzing the cytotoxicity in NIH/3T3 fibroblast cells. In both SSC7 and NIH/3T3 cell types, higher concentration of GNR induced less cell viability compared to higher concentration of GNR loaded nanocarrier. The metabolic activities of both cell types were not affected by the concentration of GNRs up to 100 μg/mL (based on the GNR amount), either by itself or as GNR loaded nanocarrier (FIG. 15a and FIG. 15b). High cell viability was observed with 250 μg/mL of GNR loaded nanocarrier, indicating that the GNR loaded nanocarrier has a favorable effect in cytotoxicity.

In vitro cellular uptake of GNR loaded nanocarrier was analyzed. SSC7 or NIH/3T3 fibroblast cells were harvested by trypsin EDTA (Gibco (Grand Island, N.Y., USA). The cells were seeded on the gelatin-coated coverslips (12 mm in diameter) in a 24-well tissue culture plate at a density of 5×10⁴ cells/well and were allowed to grow for 24 hr at 37° C. The round coverslips were sterilized by immersion them in 70% ethanol and UV exposure overnight. Then they were coated with 2% gelatin solution for optimal cell adhesion. Then, the cells were incubated with culture medium containing GNR or GNR loaded nanocarrier (50 μg/mL of GNR amount) for 2 hr to induce cellular uptake. After incubation, the cells were washed with PBS and were fixed with 4% (w/v) formaldehyde solution for 30 min, followed by two-time washing with PBS and de-ionized water. The light scattering images were recorded by using a dark field microscope (ECLIPSE L150, Nikon, Tokyo, Japan) equipped with TV lens C-0.45 camera.

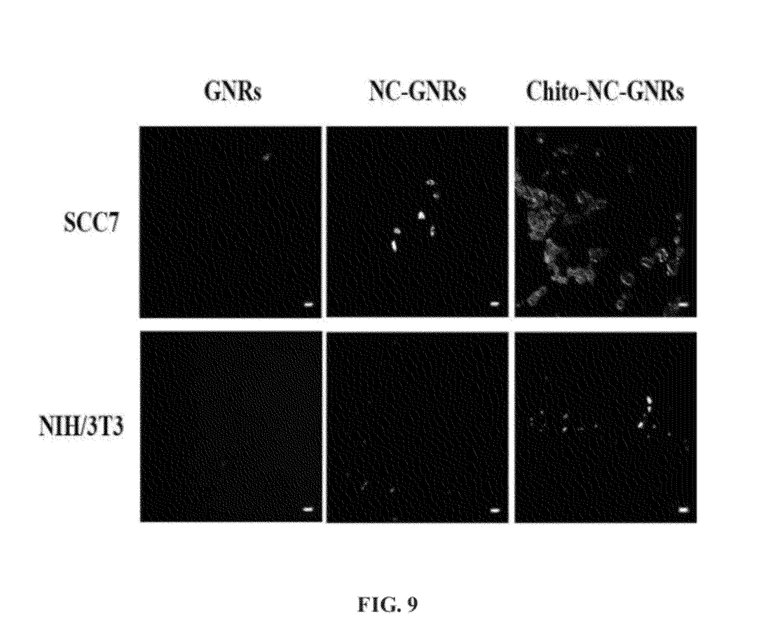

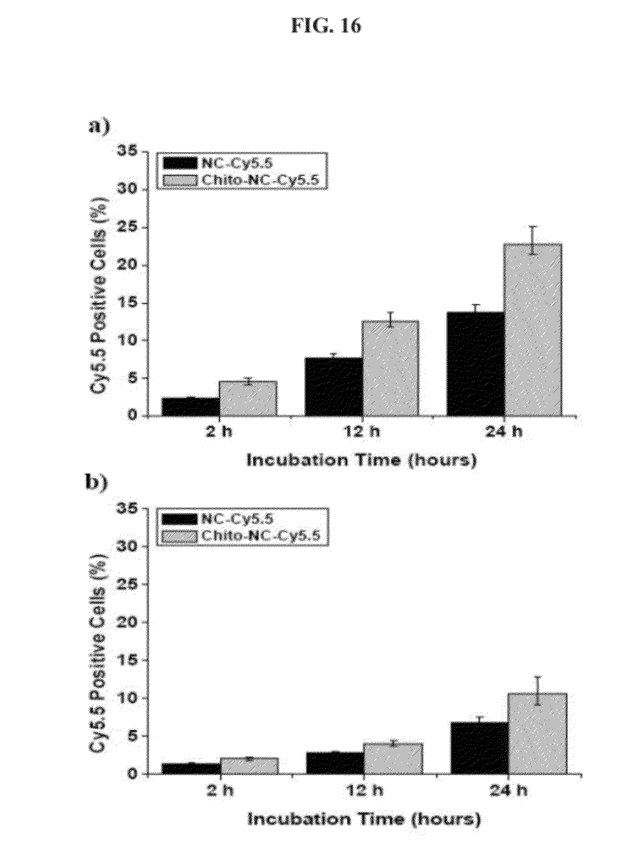

The cellular uptake of GNRs was monitored by light scattering images (50 mg/mL of GNR amount, FIG. 9). The cellular uptake was significantly increased when GNRs were encapsulated by nanocarrier. No signal was detected by direct application of GNRs, but bright spots were detected in cytosol by application of GNR loaded nanocarriers. For identical nanocarriers, the cellular uptake was higher in tumour cells than the normal fibroblast cells, suggesting that the cellular uptake of GNRs are more efficient in tumour cells than in normal cell. Also, the cellular uptake of GNRs in chitosan-modified nanocarrier was higher than the bare nanocarriers. The cellular uptake of the specific Cy5.5-labeled nanocarrier showed similar results by the laser scattering images. (FIGS. 16a and 16b)(33). As predicted, the cellular uptake of the chitosan-modified nanocarrier was significantly higher than the bare nanocarriers at each incubation time point.

The in vitro photothermal effect of GNR loaded nanocarrier was characterized. SSC7 or NIH/3T3 fibroblast cells were seeded in a 24-well tissue culture plate at a density of 8×10⁴ cells/well and were allowed to grow for 24 hr at 37° C. Then, the cells were incubated with culture medium containing 1 mL of GNR or GNR loaded nanocarrier (50 μg/mL of GNR amount). After culturing for 2 hr, the cells were washed with PBS for three times to remove non-specifically absorbed nanomaterials or nanomaterials reaming in the media. After replacing with a medium, each well was irradiated for 4 min with 780 nm laser light with 1.3 mm diameter hole-size and different power densities (41.5 and 26.4 W/cm²), by using c.a. CW Ti-sapphire laser (MIRA 900, Coherent Inc., Santa Clara, Calif., USA). Cell viability was assessed by double staining method using acridine orange (AO, Sigma-Aldrich Corp., St. Louis, Mo., USA) and propidium iodide (PI, Sigma-Aldrich Corp., St. Louis, Mo., USA). The green fluorescence of the AO indicates live cells and the red fluorescence indicates the dead cells. In brief, 1 mL of media containing 0.67 μM of AO and 75 μM of PI were added to each well, followed by 30 min incubation in dark at 37° C. After washing the cells with PBS, cell viability was visualized by inverted fluorescence microscopy (TE2000-U, Nikon, Melville, N.Y., USA)

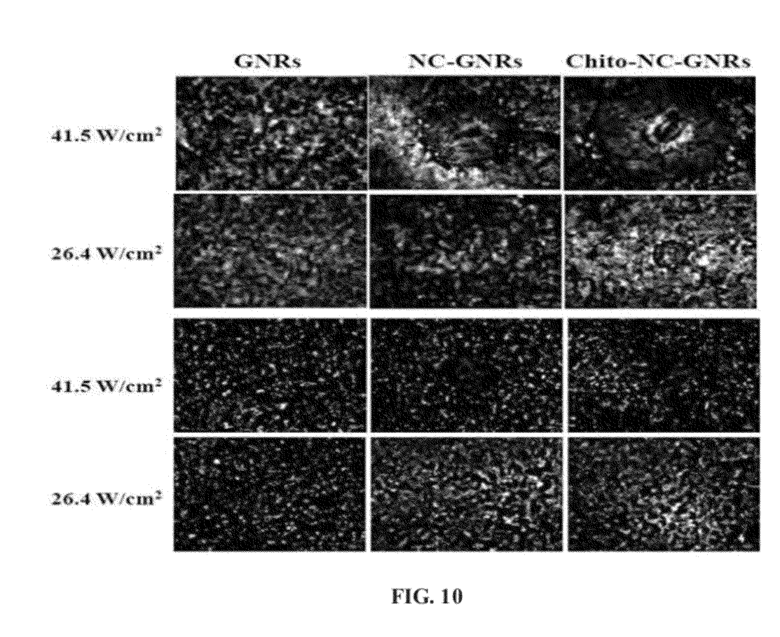

Tumour cells and fibroblast cell were treated with GNRs or GNR loaded nanocarriers (50 μg/mL of GNR amount), and then irradiated with 780 nm wavelength laser for 4 min with different power densities (41.5 and 26.4 W/cm²). The cell viability was assessed by staining with acridine orange and propidium iodide. As shown in FIG. 10a and FIG. 10b, 1) there was an improved photothermal effect when using GNR loaded nano-carries than direct application of GNRs, and no direct cellular death was observed, 2) the photothermal effect of GNR loaded nano-carries was higher in cancer cells (SCC7) than the normal cells (NIN/3T3), and 3) chitosan-modified nanocarriers showed stronger photothermal effect than the bare nanocarriers. These results correlated well with the cellular uptake results, and better outcome was observed with increased laser intensities.

### Example 7

### In Vivo Photothermal Cancer Therapy Using Chitosan-Modified Nanocarrier

All animals were obtained from Oriental Bio Co. (Seoul, Korea) and were handled in accordance with the guidelines of the Animal Care and Use Committee of Gwangju Institute of Science and Technology (GIST). To induce solid tumours, SCC7 cells (1×10⁶ in 50 μL, PBS) were injected subcutaneously on left and right side of the lumbar region. When the tumour volume reached to approximately 5 mm in diameter, GNR or GNR loaded nanocarriers (100 μg to GNR) suspended in 85% of saline solution (100 μL) were i.v. injected through the vein; saline solution was used as a control. First, in order to compare the body distribution of the nanocarriers in major organs and tumours, liver and tumours were excised from mice 24 h post i.v. injection of the nanocarriers. The tumour and liver tissue was excised and fixed in 4% formalin solution for 24 hr, before embedding in Tiusse-Tek OCT compound (Sakura Finetek, Kyoto, Japan). For cryo-sectioning, the blocks were frozen at −20° C. and sectioned. The tissue sections were stained for 10 min by using silver enhancer kit (Sigma-Aldrich Corp., St. Louis, Mo., USA) according to manufactures recommendations. The stained tissue sections were examined using inverted fluorescence microscopy. Next, to understand the photothermal ablation effect in solid tumours, mouse (left tumour: no laser irradiation vs. right tumour: laser irradiation) was NIR irradiated (808 nm diode laser, 900 mW, c.a. 4 W/cm² 5 mm beam diameter, Power Technologies, Alexander, Ark., USA) for 4 min, 24 hr post injection of nanomaterials. In addition, for further experiments, mouse was NIR irradiated for 4 min, 24 hr and 48 hr post i.v. injection. At a certain time point, the size of tumour after treatment was measured by digital caliper and the images were taken by digital camera. All measurements were performed in triplicates. Statistical analysis was performed using the Student's t-test, and for all comparisons, the minimal level of significance was set at p<0.05.

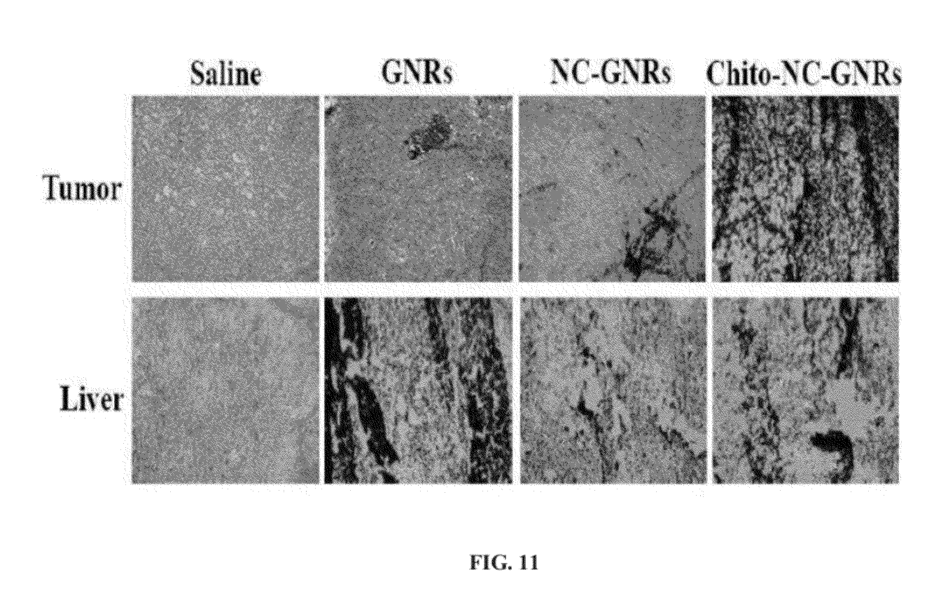

As a result, the photothermal effect on in vivo animal model was visualized by silver staining FIG. 11 is silver staining images of tumours and liver from mouse treated with GNR samples or saline solution, as a negative control. GNR loaded chitosan-modified nanocarriers showed higher intensity (dark color) in tumour cells, suggesting effective delivery into tumour cells. However, when GNR was treated directly, silver staining intensity was higher in the liver, indicating higher liver trafficking of GNRs. GNR loaded nanocarrier showed slight increase in tumour accumulation and slight decrease in liver accumulation. However, chitosan-modified nanocarrier treatment showed dramatic increase in tumour cell accumulation by silver staining method.

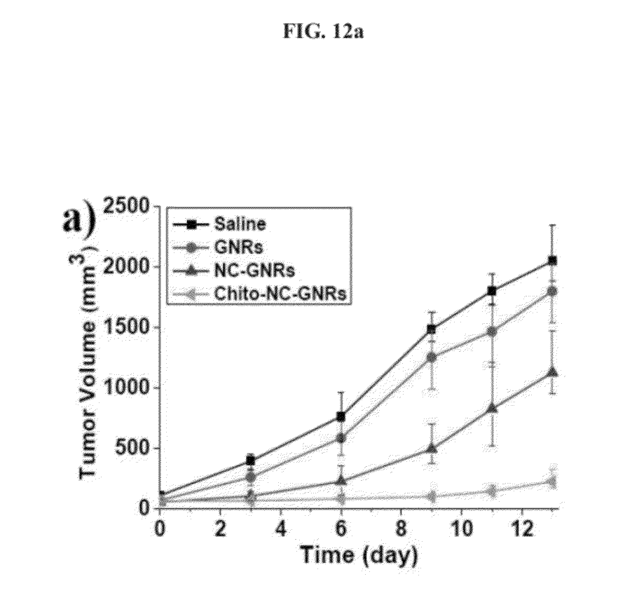

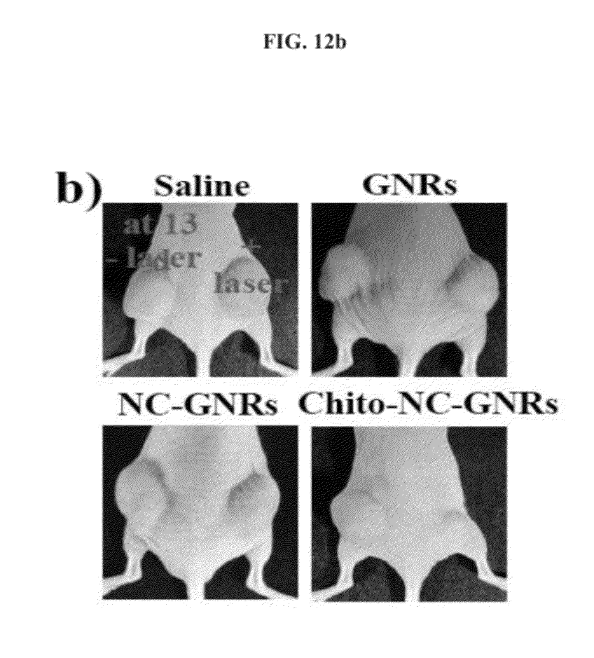

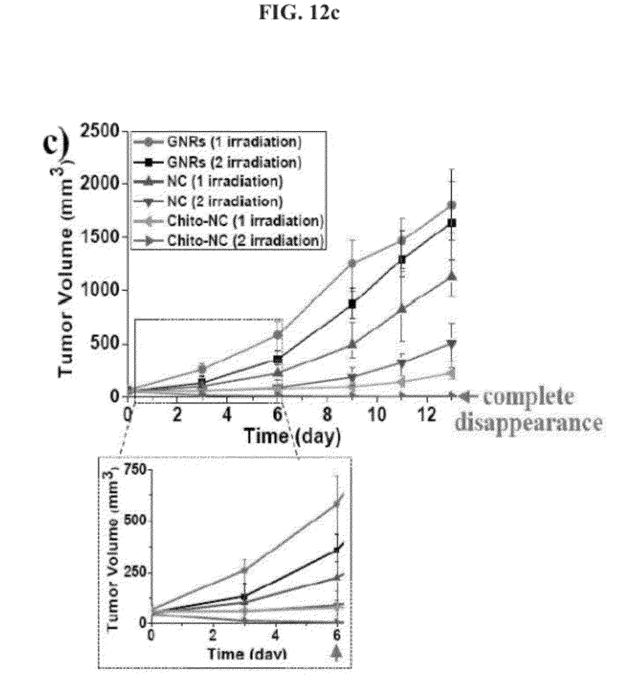

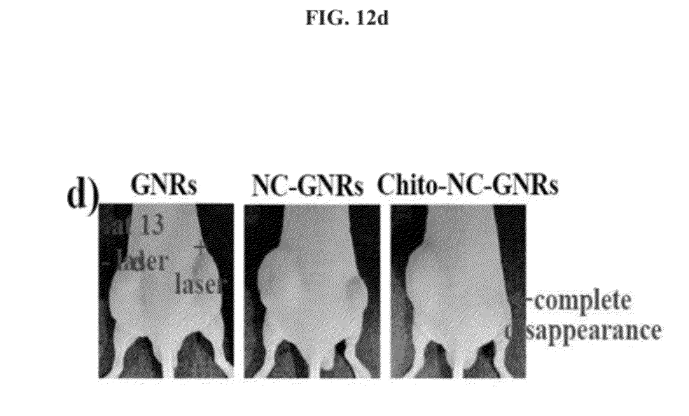

In order to analyze the therapeutic effect GNR loaded nanocarrier in photothermal ablation effect in solid tumours, mouse (left tumour: no laser irradiation vs. right tumour: laser irradiation) was NIR irradiated (808 nm, 4 W/cm²) for 4 min, 24 hr post i.v. injection. As shown in FIG. 12a-d, GNR loaded nanocarrier showed strong inhibition of tumour growth. However, direct GNR treatment showed no statistical significance in tumour regression when compared with saline treated group. As expected, chitosan-modified nanocarrier showed strong tumour growth inhibition when compared to bare form; there was no tumour volume growth for 1 week, and tumour volume increased slowly after one laser irradiation, thus clearly demonstrating the effective tumour accumulation and photothermal effect of chitosan-modified nanocarrier.

The present inventors performed additional test of NIR irradiating for twice for 4 min for possible photothermal cancer therapeutics. Laser was irradiated 24 hr and 48 hr post i.v. injection of GNR loaded nanocarriers. When laser was irradiated again at day 2, chitosan-modified nanocarrier showed complete disappearance of the tumours. Other experimental groups showed slight reduction of tumour volume after repeated laser irradiation, but direct GNR treated group did not inhibit the tumour completely (FIGS. 12c and 12d), or reduce the size (no statistical difference). More interestingly, in chitosan-modified form (Chito-NC(PF 68)) (enlarged images in FIG. 12c), there was a complete disappearance of tumour within 6 days of the initial photothermal treatment.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

### REFERENCES

1. Tong, L. et al., Gold Nanorods Mediate Tumor Cell Death by Gmpr omi s ing Membrane Integrity. Adv. Afater. 2007, 19, 3136-3141.
2. Huang, X. et al., Cancer Cell Imaging and Photothermal Ther aiy in the Near-Infrared Region by Using Gold Nanorods. 1. Am. Chem. Soc. 2006, 128, 2115-2120.
3. Chen, C. L. et al., In Situ Real-Time Investigation of Cancer Cell Photothermolysis Mediated by Exci ted Gold Nanorod Surface Pl asnons. Bjomaterjals 2010, 31, 4104-4112.
4. von Maltzahn, G. et al., SERS-Coded Gold Nanorods as a Multifunctional Platform for Densely Multiplexed Near-Infrared Imaging and Photothermal Heating. Adv. Afater. 2009, 21, 3175-3180.
5. Kuo, W. S. et al., Gold Nanorods in Photodynamic Therapy, as Hyperthermia Agents, and in Near-Infrared Optical Imaging. Angew. Chem. Int. Ed. 2010, 49, 2711-2715.
6. Gobin, A. M. et al., Near-Infrared Resonant Nanoshells for COmbined Opt i cal Imaging and Photothermal Cancer Therapy. Nano Lett. 2007, 7, 1929-1934.
7. Hu, K. W. et al., A New Photothermal Therapeutic Agent: Core-Free Nanostructured AuxAgl-x Dendrites. Chem. Eur. J. 2008, 14, 2956-2964.
8. Helmchen, F. et al., Nat. Afethods 2005, 2, 932-940.
9. Anderson, R. R.; Parrish, J. A. Selective Photothermolysis: Precise Microsurgery by Selective Absorption of Pulsed Radiation. Scjence 1983, 220, 524-527.
10. Chen, W. R. et al.; Adams, R. L.; Carubelli, R.; Nordquist, R. E. Laser-Photosensitizer Assisted Immunotherapy: A Novel Modality for Cancer Treatment. Cancer Lett. 1997, 115, 25-30.
11. Zharov, V. P. et al., Synergistic Enhancement of Selective Nanophot of hermo lys is with Gold Nanoc l ust er s: Potential for Cancer Therapy. Lasers Surg. Afed. 2005, 37, 219-226.
12. Hirsch, L. R. et al., Nanoshell-Mediated Near-Infrared Thermal Therapy of Tumors under Magnetic Resonance Guidance. Froc. Natl. Acad. Scj. USA 2003, 100, 13549-13554.
13. Loo, C. et al, Immunotargeted Nanoshells for Integrated Cancer Imaging and Therapy. Nano Lett. 2005, 5, 709-711.
14. Kim, J. et al., Designed Fabrication of Multifunctional Magtet i c Gold Nanoshells and Their App l icat ion to Magnet is Resonance Imaging and Photothermal Therapy. Angew. Chem. Int. Ed. 2006, 45, 7754-7758.
15. Chen, J. et al, Immuno Gold Nanocages with Tailored Optical Properties for Targeted Photothermal Destruct ion of Cancer Cells. Nano Lett. 2007, 7, 1318-1322.
16. Li, J. L. et al., Ultra-Low Energy Threshold for Photothermal Therapy of Cancer Using Transferrin-Conjugated Gold Nanorods. Adv. Mater. 2008, 20, 3866-3871.
17. Sau, T. K. et al., Seeded High Yield Synthesis of Short Au Nanorods in Aqueous Solution. Langmujr 2004, 20, 6414-6420.
18. Alk i l any, A. M. et al., Cellular Uptake and Cytotoxicity of Gold Nanorods: Molecular Origin of Cytotoxicity and Surface Effects. Small 2009, 5, 701-708.
19. Chakr avar ty, P. et al., Thermal Ablation of Tumor Cells with Antibody-Funct ional i zed Singl e-Walled Carbon Nanotubes. Proc, Nat l. Acad. So'. USA 2008, 105, 8697-8702.
20. Zhou, W. et al., Zwi tterionic Phosphor y l cho l Me as A Better Ligand for Gold Nanorods Cell Uptake and Selective Photothermal Ablation of Cancer Cells. Chem. Commun. 2010, 46, 1479-1481.
21. Huang, H. C. et al., Simultaneous Enhancement of Photothermal Stability and Gene Delivery Efficacy of Gold Nanorods Using Pol ye l ect ro l yt es. ACS Nano 2009, 3, 2941-2952.
22. Connor, E. E. et al., Gold Nanoparticles Are Taken Up by Hunan Cells but Do Not Cause Acute Cytotoxicity. Small 2005, 1, 325-327.
23. Huang, X. et al., Gold Nanorods: From Synthesis and Properties to Biological and Biomedical Applications. Adv. Mater. 2009, 21, 4880-4910.
24. Takahashi, H. et al., Modification of Gold Nanorods Using Phosphatidylcholine to Reduce Cytotoxicity. Langmujr 2006, 22, 2-5.
25. Hauck, T. S. et al., Assessing the Effect of Surface Chemi˜ry on Gold Nanorod Uptake, Toxicity, and Gene Expression in Mammalian Cells. Small 2008, 4, 153-159.
26. Kim, E. et al., Synthesis of Gold Nanorod-Embedded Polymeric Nanoparticles by A Nanoprecipitation Method for Use as Photothffinal Agents. Nanotechnology 2009, 20, 365602.
27. Niidome, T. et al., Po l yfethyl ene g lvco ll-Mod i f i ed Gold Namrods as A Photothermal Nanodevice for Hyperthermia. J. Bjomater. Scj. Polym. Ed. 2009, 20, 1203-1215.
28. Huang, Y. F. et al., Selective Photothermal Therapy for Mixed Cancer Cells Using Aptamer-Conjugated Nanorods. Langmujr 2008, 24, 11860-11865.
29. Huff, T. B. et al., Hyperthermic Effects of Gold Nanorods en Tumor Cells. Nanomed. 2007, 2, 125-132.
30. Li, Z. et al., RGD-Conj ugat ed Dendr imer-Modi f ied Go ld Nanorods for In Vjvo Tumor Targeting and Photothermal Therapy. Mol. Pharm. 2010, 7, 94-104. 31. Dickerson, E. B. et al., Gold Nanorod Assisted Near-InfrarEd Plasmonic Photothermal Therapy (PPTT) of Squamous Cell Carcinoma in Mice. Cancer Lett. 2008, 269, 57-66.
32. Choi, W. 1. et al., One Pot, Single Phase Synthesis of Thermo-Sens i t ive Nano-Carriers by Photo-Crosslinking of A Diacrylated Pluronic. J. Mater. Chem. 2008, 18, 2769-2774.
33. Kim, J.-Yo et al., In-Vivo Tumor Targeting of Pl ur on i c-Based Nano-Carriers. 1. Control. Release 2010, 147, 109-117.
34. Jain, P. K. et al., Calculated Absorption and Scattering Properties of Gold Nanoparticles of Different Size, Shape, and Composition: Applications in Biological Imaging and Biomedicine. J. Phys. Chem. B. 2006, 110, 7238-7248. 35. Kumar, S. et al., Plasmonic Nanosensors for Imaging Intracellular Biomarkers in Live Cells. Nano Lett. 2007, 7, 1338-1343.
36. Nikoobakht, B. et al., Preparation and Growth Mechanism of Gold Nanorods (NRs) Using Seed-Mediated Growth Method. Chem. Mater. 2003, 15, 1957-1962.
37. Iqbal, M.; Tae, G. Unstable Reshaping of Gold Nanorods Prepared by A Wet Chemical Method in the Presence of Silver Nitrate. J. Nanoscj. Nanotechnol. 2006, 6, 3355-3359.
38. Iqbal, M.; Chung, Y.-I.; Tae, G. An Enhanced Synthesis of Gold Nanorods by the Addition of Pluronic (F-127) via A Seed Mediated Growth Process. 1. Mater. Chem. 2007, 4, 335-342.
39. Chung, Y. 1. et al., The Effect of Surface Functionalizatim of PLGA Nanoparticles by Heparin- or Chitosan-Conjugated Pluronic on Tunor Targeting. J. Control. Release 2010, 143, 374-382.
40. Yang, R. et al., Enhanced Electrostatic Interaction Between Ch i tosan-Modified PLGA Nanoparticle and Tumor. Int. J. Pharm. 2009, 371, 142-147.
41 Pan, Y. et al., Size-Dependent Cytotoxicity of Gold Nanoparticles. Small 2007, 3, 1941-1949.
42. Pissuwan, D. et al., Golden Bullet? Select ive Target ing of Toxoplasma Gondii Tachyzoites Using Antibody-Functionalized Gold Nanorods. Nano Lett. 2007, 7, 3808-3812.
43. Ni idome, T. et al., PEG-Modi f ied Gold Nanorods wi th A Stealth Character for In Vjvo Applications. 1. Control. Release 2006, 114, 343-347.
44. Li, F. Q. et al., Preparation and Characterization of Sodiun Ferulate Entrapped Bovine Serum Albumin Nanoparticles for Liver Tar get i rg. Int. 1. Pharm. 2008, 349, 274-282.
45. Tae, G. et al., Formation of A Novel Heparin-Based Hydrogel in the Presence of Heparin-Binding Biomolecules. Bjomacromolecules, 2007, 8, 1979-1986.

1. A nanocarrier in which chitosan is bound to a water-soluble biocompatible polymer that has been crosslinked via a photo-crosslinkable functional group at the end; wherein the chitosan-modified nanocarrier has a diameter which changes in accordance with changes in temperature, has enhanced skin permeability, cellular uptake, selective delivery to cancer tissue or increased photothermal effect as compared with a bare nanocarrier to which chitosan has not been bound.

2. The nanocarrier of claim 1, wherein the photo-crosslinkable functional group(s) is acrylate, diacrylate, oligoacrylate, methacrylate, dimethacrylate, oligomethacrylate, coumarin, thymine or cinnamate.

3. The nanocarrier of claim 1, wherein the photo-crosslinkable functional group(s) comprises C═C double bond(s).

4. The nanocarrier of claim 1, wherein the water-soluble biocompatible polymer is starch, glycogen, chitin, peptidoglycan, lignosulfonate, tannic acid, lignin, pectin, poly(ethylene glycol), poly(ethylene oxide), poly(vinyl alcohol), poly(ethylene oxide)-poly(propylene oxide) block copolymer, cellulose, hemi cellulose, carobxymethyl cellulose, heparin, hyaluronic acid, dextran or alginate.

5. The nanocarrier of claim 4, wherein the water-soluble biocompatible polymer herein is a polymer of Formula 1:
(PC1)-(PE)ₓ-(PPO)_{y}-(PE)_{z}-(PC2) Formula 1
wherein PE is ethylene oxide; PPO is propylene oxide; each of PC1 and PC2 is a photo-crosslinkable functional group; and each of x, y and z is independently an integer of 1-10,000.

6. The nanocarrier of claim 1, wherein the average diameter of the nanocarrier increases as temperature decreases.

7. The nanocarrier of claim 1, wherein the chitosan is bound to a water-soluble biocompatible polymer that has been crosslinked via a photo-crosslinkable functional group.

8. The nanocarrier of claim 1, wherein the nanocarrier comprises a protein, a peptide, a nucleic acid, a saccharide, a lipid, a nanomaterial, a compound, an inorganic compound or a fluorescent material, or the surface of the nanocarrier is bound with a compound, an inorganic compound or a fluorescent material.

9. The nanocarrier of claim 8, wherein the protein, peptide, nucleic acid, saccharide, lipid, nanomaterial, compound or inorganic compound is a drug.

10. The nanocarrier of claim 9, wherein the drug is an anti-tumour agent.

11. The nanocarrier of claim 8, wherein the protein, peptide, nucleic acid, saccharide, lipid, compound, inorganic compound or fluorescent material is a high molecular weight material.

12. A composition for transdermal delivery comprising the nanocarrier of claim 1.

13. The composition for transdermal delivery in claim 12, wherein the nanocarrier comprises a protein, a peptide, a nucleic acid, a saccharide, a lipid, a compound or an inorganic compound with high molecular weights, or the surface of the nanocarrier is bound with a compound or an inorganic compound with high molecular weights.

14. The composition for transdermal delivery in claim 13, wherein the protein, the peptide, the nucleic acid, the saccharide, the lipid, the compound or the inorganic compound with high molecular weight is a drug.

15. A composition for in vivo tumor or cancer imaging comprising the nanocarrier of claim 1.

16. A composition for photothermal cancer therapy comprising the nanocarrier of claim 1.

17. A cargo delivery method comprising a step of contacting a subject's skin with the nanocarrier of claim 1 comprising a cargo material.

18. A transdermal cargo delivery method comprising a step of contacting a subject's skin with the nanocarrier of claim 1 comprising a cargo material.

19. The method of claim 18, wherein the cargo material comprises a protein, a peptide, a nucleic acid, a saccharide, a lipid, a compound or an inorganic compound high molecular weights, or the surface of the cargo material is bound with a compound or an inorganic compound with high molecular weights.

20. The method of claim 19, wherein the protein, the peptide, the nucleic acid, the saccharide, the lipid, the compound or the inorganic compound with high molecular weights is a drug.

21. A method for in vivo tumour or cancer imaging of a subject, which comprises the steps of:
(a) administering a diagnostically effective dose of the nanocarrier of claim 1 comprising the cargo material to the subject; and
(b) acquiring visible images by scanning the subject.

22. A method for photothermal cancer therapy comprising a step of administering a therapeutically effective dose of the nanocarrier of claim 1 comprising the cargo material to the subject.

23. A process of preparing chitosan-modified nanocarrier having a diameter which changes in accordance with changes in temperature, has enhanced skin permeability or cellular uptake compared with a bare nanocarrier to which chitosan has not been bound, which comprises the steps of:
(a) preparing a dispersion comprising a water-soluble biocompatible polymer with photo-crosslinkable functional group;
(b) preparing a dispersion comprising a water-soluble natural polymer with photo-crosslinkable functional group;
(c) preparing a mixture of the dispersion comprising biocompatible polymer and the dispersion comprising chitosan;
(d) adding an initiator to the mixture; and
(e) preparing a chitosan-modified nanocarriers by crosslinking the polymer and chitosan by irradiating light onto the product of step (d).

24. The process of claim 23, wherein the photo-crosslinkable functional group(s) is acrylate, diacrylate, oligoacrylate, methacrylate, dimethacrylate, oligomethacrylate, coumarin, thymine or cinnamate.

25. The process of claim 23, wherein the photo-crosslinkable functional group(s) comprises C═C double bond(s).

26. The process of claim 23, wherein the water-soluble biocompatible polymer is starch, glycogen, chitin, peptidoglycan, lignosulfonate, tannic acid, lignin, pectin, poly(ethylene glycol), poly(ethylene oxide), poly(vinyl alcohol), poly(ethylene oxide)-poly(propylene oxide) block copolymer, cellulose, hemi cellulose, carobxymethyl cellulose, heparin, hyaluronic acid, dextran or alginate.

27. The process of claim 26, wherein the water-soluble biocompatible polymer herein is a polymer of Formula 1:
(PC1)-(PE)ₓ-(PPO)_{y}-(PE)_{z}-(PC2) Formula 1
wherein PE is ethylene oxide; PPO is propylene oxide; each of PC1 and PC2 is a photo-crosslinkable functional group; and each of x, y and z is independently an integer of 1-10,000.

28. The process of claim 23, wherein the light of step (e) is an UV light.

29. The process of claim 23, wherein the average diameter of the chitosan-modified nanocarrier increases as temperature decreases.

30. The process of claim 23, wherein the steps of (a)-(e) are carried out in an aqueous dispersion phase without using an organic dispersion phase.

31. The process of claim 23, wherein the pore size of the chitosan-modified nano-carrier at 37° C. is between 3 and 20 nm.
